(19) United States
(12) Patent Application Publication
(10) Pub. No.: US 2009/0012475 A1
(43) Pub. Date: Jan. 8, 2009

(54) MEDICAL GUIDE WIRE

(75) Inventors: Yoshio Onuki, Hino-shi (JP); Koh Kimura, Sagamihara-shi (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, Tokyo (JP)

(21) Appl. No.: 12/146,713

(22) Filed: Jun. 26, 2008

Related U.S. Application Data
(63) Continuation of application No. 10/657,670, filed on Sep. 8, 2003, now abandoned.
Application No. 10/657,670 is a division of application No. 10/059,681, filed on Jan. 30, 2002, now abandoned.

(30) Foreign Application Priority Data
May 30, 2000 (JP) 2000160301
May 17, 2001 (JP) PCT/JP01/04126

(51) Int. Cl. A61M 25/09

(52) U.S. Cl. 604/164.13; 600/106

(57) ABSTRACT

A fixing portion is provided so that the position of a guide wire (1) relative to an endoscope (2) is fixed lest it change by means of a retaining wire (12) that has one end coupled to the distal end portion side of a guide wire body (11) and the other end extending to the proximal end portion side of the guide wire body (11).

### CROSS REFERENCE TO RELATED APPLICATIONS

This application is a continuation application of U.S. Ser. No. 10/657,670 filed on Sep. 8, 2003 which is a divisional application of U.S. Ser. No. 10/059,681 filed on Jan. 30, 2002, the contents of each of which are incorporated herein by reference.

### TECHNICAL FIELD

The present invention relates to a medical guide wire for guiding an appliance to be passed through a channel of an endoscope and inserted into the human body in insertion operation, in endoscopy or endoscopic operations on the pancreatic or biliary duct system, in particular.

### BACKGROUND ART

Recently, there have been increasing endoscopic treatments in which diseases in the digestive tract system and pancreatic or biliary duct system are treated by means of an endoscope. Existing treatments on the pancreatic or biliary duct system using an endoscope include therapeutic treatments in which gallstones in the common bile duct, for example, are recovered by means of a balloon or holding forceps, as well as diagnostic treatments in which the biliary duct and pancreatic duct are visualized endoscopically.

Usually, in performing an endoscopic treatment on the pancreatic, biliary, or hepatic duct by means of an endoscope, the distal end portion of the insert section of the endoscope is inserted into a region near the duodenal papilla. Then, an appliance such as a catheter is selectively inserted into the pancreatic or biliary duct with a guide wire used as a guide in radioscopy.

More specifically, the following operations are carried out. First, a distal end portion c of an insert section b of an endoscope a shown in FIGS. 55A and 555 is inserted into a region near the duodenal papilla. Thereafter, a catheter d is inserted into an appliance passage channel of the endoscope a. As this is done, a distal end portion d1 of the catheter d is inserted into the pancreatic or biliary duct through the endoscope. Then, a guide wire e is inserted through a mouthpiece d2 on the proximal end side of the inserted catheter d.

Thereafter, it is confirmed by means of X-rays that the guide wire e is correctly inserted in the pancreatic or biliary duct. Subsequently, the proximal end side of the guide wire e is manually held as the catheter d is drawn out of the appliance passage channel of the endoscope a, as shown in FIG. 55A. When the distal end portion d1 of the catheter d emerges from a forceps port g on the side of an operating section f of the endoscope a during this operation, as shown in FIG. 55B, the whole catheter d is entirely drawn out of the endoscope a in a manner such that the guide wire e is manually held in a position near the forceps port g of the endoscope a.

Then, the proximal end side of the guide wire e is inserted into a passage hole of another appliance, and the alternative appliance is guided by means of the guide wire e as it is inserted into the appliance passage channel of the endoscope a. Thereafter, the aforementioned operations are repeated for each replacement of an appliance.

In general, the catheter d and some other appliances used in these treatments are given lengths of 1,900 mm in consideration of the length of the insert section b of the endoscope a. In order to the replace the appliance in the aforesaid steps of the procedure, the length of an extended portion of the guide wire e that extends outward from the forceps port g on the side of the operating section f of the endoscope a should not be shorter than the length of the catheter d when the distal end portion of the guide wire e is caused to project for a given length from the appliance passage channel of the endoscope a (e.g., when the distal end of the guide wire e is inserted in the pancreatic or biliary duct), as shown in FIG. 55A. Thus, the overall length of the guide wire e should not be shorter than the sum of the respective lengths of the insert section b of the endoscope a and the catheter d or some other appliance, so that it is expected to be at least about 4,000 mm.

Described in U.S. Pat. No. 5,921,971, for example, is a catheter in which a longitudinal opening (slit) is formed extending between the distal and proximal end portions of a guide wire lumen of a catheter shaft so that a replacement operation can be carried out using a short guide wire.

In observing or treating the pancreatic or biliary duct system by means of the endoscope a, the guide wire e is inserted in the catheter d or some other appliance in the case where the appliance is passed through the appliance passage channel of the endoscope a. If the appliance is moved relatively to the endoscope a, therefore, the guide wire e inevitably moves at the same time. In replacing the appliance guided by means of the guide wire e with the distal end of the guide wire e inserted in the papilla, for example, therefore, the guide wire e must be always held on the side of the operating section f of the endoscope a, in order to keep the distal end of the guide wire e inserted in the papilla.

In replacing the appliance during the use of the endoscope a with the conventional configuration, moreover, two operations must be simultaneously carried out such that the appliance is drawn out of the appliance passage channel of the endoscope a as the guide wire a is inserted for the same distance of movement or that the appliance is inserted into the appliance passage channel in like manner as the guide wire e is drawn out for the same distance of movement. Thus, the manipulation is complicated and troublesome.

Since the guide wire e is as long as about 4,000 mm, moreover, it is hard to handle the guide wire e so as not to allow it to touch any dirty region, such as the floor in a narrow endoscope chamber. Since the appliance cannot be replaced unless it is moved for a distance corresponding to the overall length of the guide wire e, furthermore, the replacement of the appliance itself takes a long time. Accordingly, the operation for replacing the endoscopic appliance inevitably requires a lot of time.

Further, the operation for replacing the endoscopic appliance requires the presence of at least two assistants in an operating room. Therefore, much manpower cost is required, which inevitably increase the financial burdens on hospitals and patients.

In the case of the catheter described in U.S. Pat. No. 5,921,971 arranged so that the longitudinal opening (slit) is formed extending between the distal and proximal end portions of the guide wire lumen of the catheter shaft, moreover, operation is needed to provide a conventional contrastradiography catheter with the opening (slit). Accordingly, its manufacturing cost is inevitably higher than the conventional contrastradiography catheter.

In order to compensate for the reduction in stiffness of the catheter shaft that is attributable to the formation of the slit, moreover, the outside diameter of the shaft must be increased or a more rigid material must be used for the shaft. Thus, the increase of the shaft diameter worsens the ease of insertion in the channel of the endoscope, which possibly lowers the operational efficiency.

Treatments on the pancreatic and biliary duct systems require veteran skill, and a large number of techniques are available. Therefore, operators are especially particular about their appliances. Further, the condition of the patient also affects the way the appliances are used. According to this prior art, however, the number of available appliances is inevitably limited, which leaves little choice for the operator.

The present invention has been contrived in consideration of these circumstances, and its object is to provide a medical guide wire with which an endoscopic appliance can be replaced speedily and easily without interfering with the conventional method of endoscopic appliance operation or the sense of operation.

Further, another object is to provide a medical guide wire designed so that the guide wire can be securely fixed by means of a guide wire fixing mechanism that is composed of a forceps raising block and a guide wire fixture arranged on the distal end of an insert section of an endoscope, so that an endoscopic appliance can be replaced speedily and easily.

### DISCLOSURE OF INVENTION

The present invention is intended to provide a guide wire with a mechanism for fixing the guide wire to an endoscope without the necessity of holding the proximal end portion side of the guide wire.

The following is a description of a specific configuration.

According to the present invention, there is provided a medical guide wire which comprises a guide wire body to be passed through a channel of an endoscope, the guide wire body serving to guide an appliance to be inserted into the human body in insertion operation, the medical guide wire comprising a fixing portion formed of a substantially wire-shaped retainer having one end coupled to the distal end portion side of the guide wire body and the other end extending to the proximal end portion side of the guide wire body and used to fix the position of the medical guide wire by means of the retainer lest the position of the medical guide wire relative to the endoscope change.

According to the present invention, moreover, one end of the substantially wire-shaped retainer is coupled to the distal end portion side of the guide wire body, and the wire-shaped retainer extends parallel to the guide wire body and close to the hand-side end of the guide wire body on its proximal end portion side. In inserting or removing the appliance into the appliance passage channel of the endoscope through the guide wire body, therefore, the guide wire body can be fixed by holding the proximal end portion side of the wire-shaped retainer in a manner such that the distal end portion of the guide wire body projects for a given length from the channel of the endoscope. Since the appliance can be inserted or removed in this state, the length of the guide wire body itself can be made shorter, and the appliance can be replaced in a shorter time and more easily. Further, the manpower cost can be lowered since only one or no assistant is required by the operation for replacing the endoscopic appliance. Since the configuration on the appliance side need not be changed at all, moreover, the appliance replacement operation can be easily carried out without interfering with the conventional operating method or the sense of operation.

According to the present invention, there is provided a medical guide wire comprising a guide wire body to be passed through a channel of an endoscope, the guide wire body serving to guide an appliance to be inserted into the human body in insertion operation, the guide wire body being provided with an engagement aiding portion on the distal end portion side thereof, adapted releasably to engage a guide wire fixing mechanism on the side of a distal end opening of the channel of the endoscope, thereby aiding engagement with the guide wire fixing mechanism, when the distal end portion of the guide wire body is detachably anchored by means of the guide wire fixing mechanism.

According to the present invention, moreover, higher fixing strength can be obtained in a manner such that the engagement aiding portion on the distal end portion side of the guide wire body is caused releasably to engage the guide wire fixing mechanism on the distal end opening side of the channel of the endoscope, thereby aiding engagement with the guide wire fixing mechanism, when the distal end portion of the guide wire body is held and detachably anchored by means of the guide wire fixing mechanism.

### BRIEF DESCRIPTION OF DRAWINGS

FIG. 1 is a perspective view showing a state of use of a medical guide wire of a first embodiment of the present invention;

FIG. 2 is a side view showing the distal end portion of the medical guide wire of the first embodiment;

FIG. 3A is a longitudinal sectional view of the medical guide wire of the first embodiment;

FIG. 3B is a sectional view taken along line 3B-3B of FIG. 3A;

FIG. 4A is a side view showing the way an endoscopic appliance is passed with the medical guide wire of the first embodiment used as a guide;

FIG. 4B is a sectional view taken along line 4B-4B of FIG. 4A;

FIG. 5 is a diagram for illustrating replacement operation for an endoscopic appliance by means of the medical guide wire of the first embodiment;

FIG. 6 is a diagram for illustrating operation for inserting the endoscopic appliance, inserted into a channel of an endoscope by using the medical guide wire of the first embodiment, into the body cavity;

FIG. 7 is a longitudinal sectional view of a principal part showing a modification of the medical guide wire of the first embodiment;

FIG. 8A is a longitudinal sectional view of a principal part showing a medical guide wire of a second embodiment of the present invention;

FIG. 8B is a longitudinal sectional view of a principal part showing the medical guide wire combined with the endoscopic appliance;

FIG. 9 is a longitudinal sectional view of a medical guide wire showing a third embodiment of the present invention;

FIG. 10 is a longitudinal sectional view of a medical guide wire showing a fourth embodiment of the present invention;

FIG. 11 is a side view of a medical guide wire showing a fifth embodiment of the present invention;

FIG. 12 is a side view of a medical guide wire showing a sixth embodiment of the present invention;

FIG. 13A is a side view showing the distal end portion of a medical guide wire of a seventh embodiment of the present invention;

FIG. 13B is a plan view of the same portion;

FIG. 13C is a sectional view taken along line 13C-13C of FIG. 13B;

FIG. 14 is a diagram for illustrating a state of use of the medical guide wire of the seventh embodiment;

FIG. 15 is a longitudinal sectional view of a principal part showing the distal end portion of a medical guide wire of an eighth embodiment of the present invention;

FIG. 16 is a side view of a principal part showing the distal end portion of a medical guide wire of a ninth embodiment of the present invention;

FIG. 17 is a side view of a principal part showing a state of insertion of a drainage tube by means of a medical guide wire of a tenth embodiment of the present invention;

FIG. 18 is a sectional view taken along line 18-18 of FIG. 17;

FIG. 19 is a diagram for illustrating operation for inserting the drainage tube, inserted into the channel of the endoscope by using the medical guide wire of the tenth embodiment, into the body cavity;

FIG. 20A is a perspective view showing a state of use of a medical guide wire of an eleventh embodiment of the present invention;

FIG. 20B is a perspective view showing a fixing portion for a medical guide wire;

FIG. 21 is a perspective view of a principal part showing a twelfth embodiment of the present invention;

FIG. 22 is a diagram for illustrating the way a medical guide wire of a thirteenth embodiment of the present invention is used in combination with an endoscope;

FIG. 23A is a plan view of the distal end portion of an insert section showing a state before a forceps raising block is raised as the medical guide wire of the thirteenth embodiment is raised;

FIG. 23B is a longitudinal sectional view of the same portion;

FIG. 23C is a plan view of the distal end portion of the insert section showing the guide wire held and fixed between the forceps raising block and a guide wire fixing member;

FIG. 23D is a longitudinal sectional view of the same portion;

FIG. 24 is a side view showing the distal end portion of the medical guide wire of the thirteenth embodiment;

FIG. 25A is a longitudinal sectional view of the medical guide wire of the thirteenth embodiment;

FIG. 25B is a sectional view taken along line 25B-25B of FIG. 25A;

FIG. 26A is a plan view showing an engagement aiding portion of the medical guide wire of the thirteenth embodiment;

FIG. 26B is a sectional view taken along line 26B-26B of FIG. 26A;

FIG. 27 is a diagram for illustrating a state of use of the engagement aiding portion of the medical guide wire of the thirteenth embodiment;

FIG. 28 is a longitudinal sectional view of a principal part showing a modification of the medical guide wire of the thirteenth embodiment;

FIG. 29 is a longitudinal sectional view of a principal part showing another modification of the medical guide wire of the thirteenth embodiment;

FIG. 30A is a side view showing a preshaped portion of a medical guide wire of a fourteenth embodiment of the present invention;

FIG. 30B is a side view showing a modification of the preshaped portion of the medical guide wire;

FIG. 31 is a diagram for illustrating a state of use of the medical guide wire of the fourteenth embodiment;

FIG. 32 is a perspective view of a principal part showing the distal end portion of a medical guide wire of a fifteenth embodiment of the present invention;

FIG. 33 is a diagram for illustrating a state of use of the medical guide wire of the fifteenth embodiment;

FIG. 34A is a plan view showing a guide wire fixed by means of a guide wire fixing mechanism of an endoscope of a sixteenth embodiment of the present invention;

FIG. 34B is a perspective view showing an engaging groove of a forceps raising block;

FIG. 35 is a perspective view of a principal part showing the distal end portion of a medical guide wire of a seventeenth embodiment of the present invention;

FIG. 36A is a longitudinal sectional view of a principal part showing a guide wire sheath of a medical guide wire of an eighteenth embodiment of the present invention held in a standby position;

FIG. 36B is a longitudinal sectional view of a principal part showing the guide wire sheath moved to an advanced position;

FIG. 37A is a plan view of a principal part showing the distal end portion of a medical guide wire according to a nineteenth embodiment of the present invention;

FIG. 37B is a side view of the same part;

FIG. 37C is a sectional view taken along line 37C-37C of FIG. 37A;

FIG. 37D is a sectional view taken along line 37D-37D of FIG. 37A;

FIG. 38 is a plan view showing the medical guide wire of the nineteenth embodiment fixed by means of the guide wire fixing mechanism of the endoscope;

FIG. 39 is a plan view of a principal part showing the distal end portion of a medical guide wire of a twentieth embodiment of the present invention;

FIG. 40 is a plan view of a principal part showing the distal end portion of a medical guide wire of a twenty-first embodiment of the present invention;

FIG. 41 is a plan view of a principal part showing the distal end portion of a medical guide wire of a twenty-second embodiment of the present invention;

FIG. 42 is a plan view of a principal part showing the distal end portion of a medical guide wire of a twenty-third embodiment of the present invention;

FIG. 43A is a plan view of a principal part showing the distal end portion of a medical guide wire of a twenty-fourth embodiment of the present invention;

FIG. 43B is a longitudinal sectional view of a principal part showing a first modification of the medical guide wire of the twenty-fourth embodiment;

FIG. 43C is a longitudinal sectional view of a principal part showing a second modification of the medical guide wire of the twenty-fourth embodiment;

FIG. 44 is a longitudinal sectional view of a principal part showing the distal end portion of a medical guide wire of a twenty-fifth embodiment of the present invention;

FIG. 45 is a perspective view of a principal part showing the distal end portion of a medical guide wire of a twenty-sixth embodiment of the present invention;

FIG. 46 is a longitudinal sectional view of a principal part showing the distal end portion of a medical guide wire of a twenty-seventh embodiment of the present invention;

FIG. 47A is a diagram for illustrating a state of use of the medical guide wire of the twenty-seventh embodiment;

FIG. 47B is a side view of a principal part showing a modification of the medical guide wire;

FIG. 48 is a longitudinal sectional view of a principal part showing the distal end portion of a medical guide wire of a twenty-eighth embodiment of the present invention;

FIG. 49 is a longitudinal sectional view of a principal part showing the distal end portion of a medical guide wire of a twenty-ninth embodiment of the present invention;

FIG. 50 is a perspective view of a principal part showing the distal end portion of a medical guide wire of a thirtieth embodiment of the present invention;

FIG. 51 is a longitudinal sectional view of a principal part showing the distal end portion of a medical guide wire of a thirty-first embodiment of the present invention;

FIG. 52 is a longitudinal sectional view of a principal part showing the distal end portion of a medical guide wire of a thirty-second embodiment of the present invention;

FIG. 53 is a longitudinal sectional view of a principal part showing the distal end portion of a medical guide wire of a thirty-third embodiment of the present invention;

FIG. 54 is a side view of a principal part showing the distal end portion of a medical guide wire of a thirty-fourth embodiment of the present invention;

FIG. 55A is a diagram for illustrating the way a catheter is drawn out of the appliance passage channel of the endoscope by using a guide wire as an endoscopic treatment is performed by means of the endoscope by a conventional method; and

FIG. 55B is a diagram for illustrating the way the catheter is entirely drawn out of the endoscope.

### BEST MODE FOR CARRYING OUT OF THE INVENTION

A first embodiment of the present invention will now be described with reference to FIGS. 1 to 6. FIG. 1 shows a state in which a medical guide wire 1 of the present embodiment and an endoscope 2 are used in combination. The endoscope 2 is provided with an elongate insert section 3 to be inserted into the body cavity, a hand-side operating section 4 coupled to the proximal end portion of the insert section 3, and a universal cord (not shown) to which the proximal end portion of the operating section 4 is coupled. Further, the insert section 3 is provided with components that include an elongate flexible tube portion 5 having flexibility, a curved portion 6 coupled to the distal end of the flexible tube portion 5, and a distal end portion 7 located in the extreme end position of the insert section 3.

An appliance passage channel (not shown) for use as an appliance passage guide way is located in the insert section 3 of the endoscope 2. The distal end portion 7 of the insert section 3 is formed having a channel opening 8 that constitutes a distal end opening of the appliance passage channel. Further, the hand-side operating section 4 is provided with an appliance inlet portion 9 that communicates with the proximal end portion of the appliance passage channel. The endoscopic appliance such as a catheter 10 is inserted into the appliance passage channel through the hand-side operating section 4 and guided to the side of the distal end portion 7 of the insert section 3 through the appliance passage channel. Thereafter, it projects outward through the channel opening 8 of the distal end portion 7.

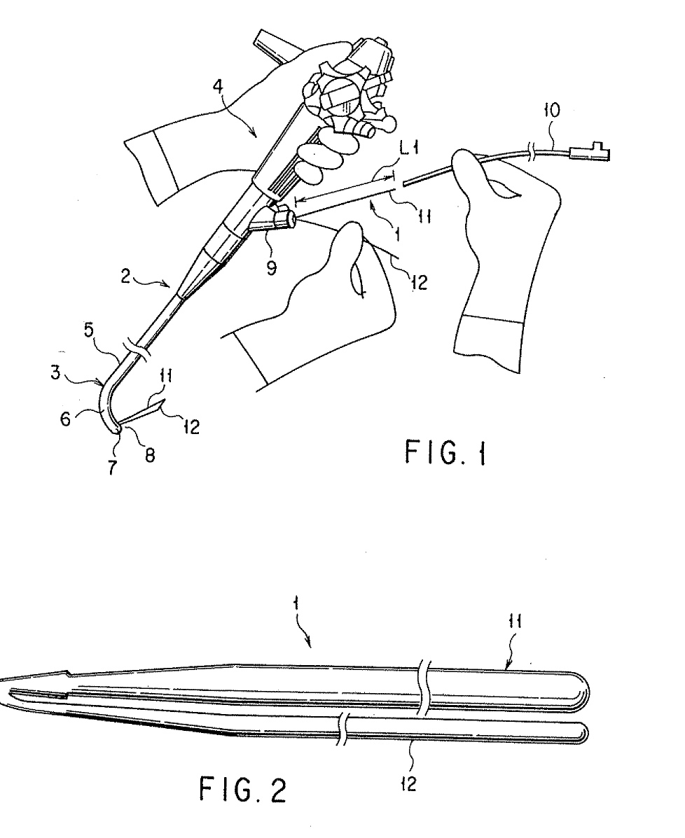

In the medical guide wire 1 of the present embodiment, moreover, the distal end portion of a retaining wire (substantially wire-shaped retainer) 12 is coupled to the distal end portion side of a guide wire body 11, as shown in FIG. 2. The proximal end portion of the retaining wire 12 extends parallel to the guide wire body 11 and close to the hand-side end of the proximal end portion side of the guide wire body 11.

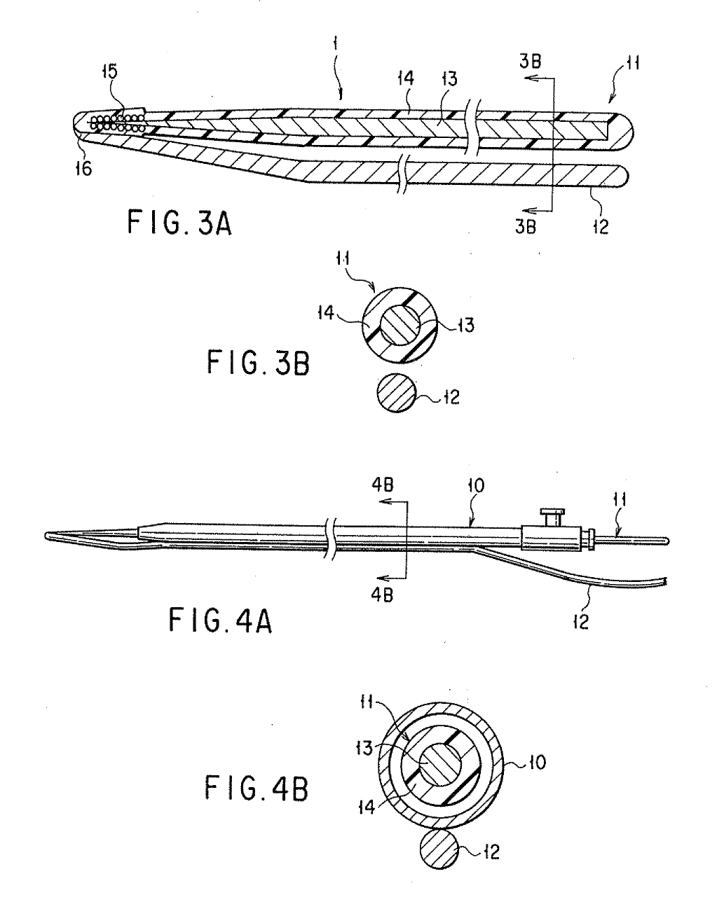

In the guide wire body 11, as shown in FIGS. 3A and 3B, a coating layer 14 of a plastic material such as fluoroplastic or polyurethane is provided around an elongate tapered core 13. Further, an X-ray marker 15 is attached to the distal end portion of the core 13. The X-ray marker 15 is formed by tightly winding a wire of an X-ray-nonpermeable material, such as platinum, gold, silver, palladium, tantalum, or tungsten that does not transmit X-rays, around the distal end portion of the core 13.

The guide wire body 11 is not limited to a single wire, and may be formed of multiple strands or a closely-wound coil, or be a guide wire of any other known form. Further, the length of the guide wire body 11 is adjusted to about 2,300 to 2,600 mm, for example, and the wire diameter to about 0.9 mm, for example.

The retaining wire 12 is formed of a single wire or multiple strands of a superelastic alloy, such as a nickel-titanium alloy, stainless steel, iron, amorphous metal, various alloys such as a titanium alloy, nickel alloy, and cobalt alloy, carbon fiber, relatively rigid plastic materials, etc. Further, the wire diameter of the retaining wire 12 is adjusted to about 0.2 to 0.5 mm, for example, and its length to about 2,300 to 2,600 mm, for example. The wire diameter of the retaining wire 12 is not limited to this, and may be suitably set at a value that matches the diameter of the catheter 10 or some other endoscopic appliance, the wire diameter of the guide wire body 11, and the inside diameter of the appliance passage channel of the endoscope 2, such that the wire 12 can be passed through the appliance passage channel.

The distal end portion of the retaining wire 12 is bonded to the distal end portion of the guide wire body 11 by bonding means such as adhesive bonding or solvent welding, and is coupled to the distal end portion of the guide wire body 11 by means of this bond portion 16.

The following is a description of the function of the configuration described above. In working the medical guide wire 1 of the present embodiment, the guide wire body 11 is previously inserted into the tube bore of the catheter 10 or some other endoscopic appliance, as shown in FIGS. 4A and 4B. As this is done, the catheter 10 or some other endoscopic appliance is set in a state such that it is inserted in a position near the distal end portion of the guide wire body 11.

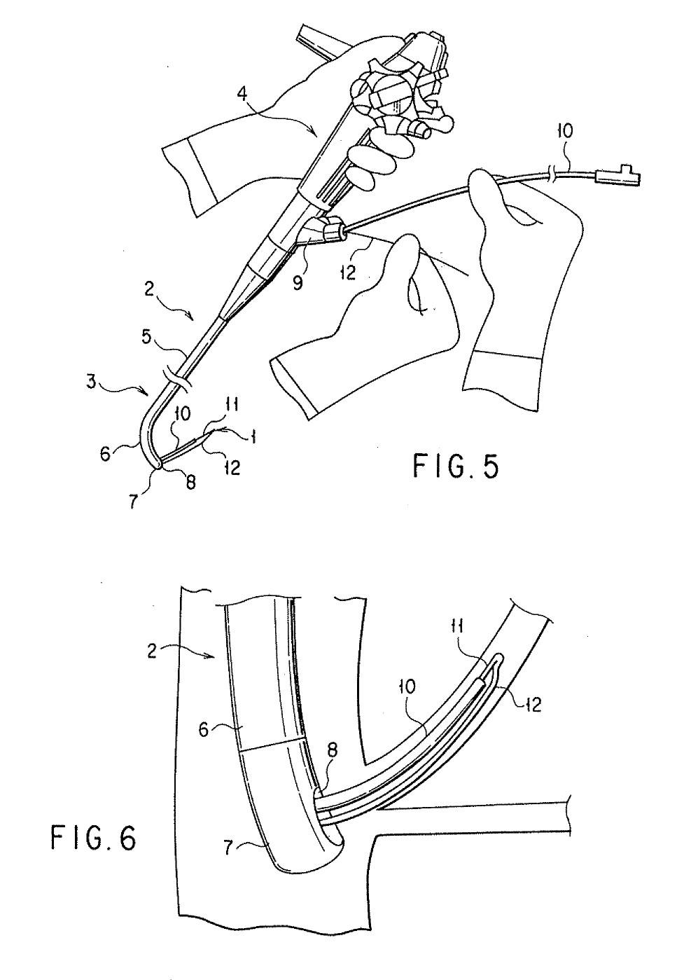

In this state, the catheter 10, along with the medical guide wire 1 of the present embodiment, is inserted into the appliance passage channel through the appliance inlet portion 9 of the operating section 4 of the endoscope 2. Then, the catheter 10 is caused to project outward through the channel opening 8 of the distal end portion 7 of the insert section 3, as shown in FIG. 5, and is inserted into the pancreatic or biliary duct per papilla, as shown in FIG. 6.

Thereafter, the following operation is carried out to replace the currently engaged catheter 10 with an appliance to be used next. First, the proximal end portion side of the retaining wire 12 is manually held with the distal end portion of the guide wire body 11 kept projecting for a given length from the channel of the endoscope 2, as shown in FIG. 5. Thereupon, the guide wire body 11 is fixed to prevent it being moved. Subsequently, in this state, an operation is carried out to pull out the catheter 10, and the catheter 10 is entirely drawn out of the appliance passage channel through the appliance inlet portion 9 on the side of the operating section 4 of the endoscope 2. As this is done, the catheter 10 is drawn out from the proximal end portion side of the guide wire body 11, as shown in FIG. 1. In this case, a length L1 for which the guide wire body 11 extends outward from the appliance inlet portion 9 must only range from tens of millimeters to hundreds of millimeters, as shown in FIG. 1. Therefore, the overall length of the guide wire body 11 is good enough if it ranges from about 2,300 to 2,600 mm. The same applies to the length for which the retaining wire 12 extends from the appliance inlet portion 9 and its overall length.

Thereafter, the distal end portion of the guide wire body 11 is caused to project for the given length from the channel of the endoscope 2, and the appliance to be used next is inserted through the proximal end side of the guide wire body 11 with the proximal end portion side of the retaining wire 12 held manually. With the guide wire body 11 used as a guide, the appliance is inserted into the appliance passage channel through the appliance inlet portion 9 on the side of the operating section 4 of the endoscope 2. Then, the appliance is caused to project outward through the channel opening 8 of the distal end portion 7 and further inserted into the pancreatic or biliary duct. Thereupon, the replacement of the appliance is finished. According to FIGS. 1 and 5, an operator who operates the endoscope 2 holds the proximal end portion side of the retaining wire 12 in his/her hand, and another person or an assistant inserts or removes the endoscopic appliance. Alternatively, however, the operator may insert or remove the endoscopic appliance with the other hand while holding the retaining wire 12 in the same hand that holds the endoscope 2.

The configuration described above has the following effects. In the medical guide wire 1 of the present embodiment, one end of the retaining wire 12 is coupled to the distal end portion side of the guide wire body 11, while the retaining wire 12 extends parallel to the guide wire body 11 and close to the hand-side end of the guide wire body 11 on its proximal end portion side. In inserting or removing the catheter 10 or some other appliance into the appliance passage channel of the endoscope 2 through the guide wire body 11, therefore, the guide wire body 11 can be fixed by holding the proximal end portion side of the retaining wire 12 in a manner such that the distal end portion of the guide wire body 11 projects by the given length from the channel of the endoscope 2. Since the catheter 10 or some other appliance can be inserted or removed in this state, the guide wire body 11 itself need only be as long as 2,300 to 2,600 mm. Thus, the guide wire body 11 itself can be made shorter than a conventional one, and the appliance can be replaced in a shorter time and more easily. Further, the manpower cost can be lowered since only one or no assistant is required by the operation for replacing the endoscopic appliance. Since the configuration on the appliance side need not be changed at all, moreover, the appliance replacement operation can be easily carried out without interfering with to the conventional operating method or the sense of operation.

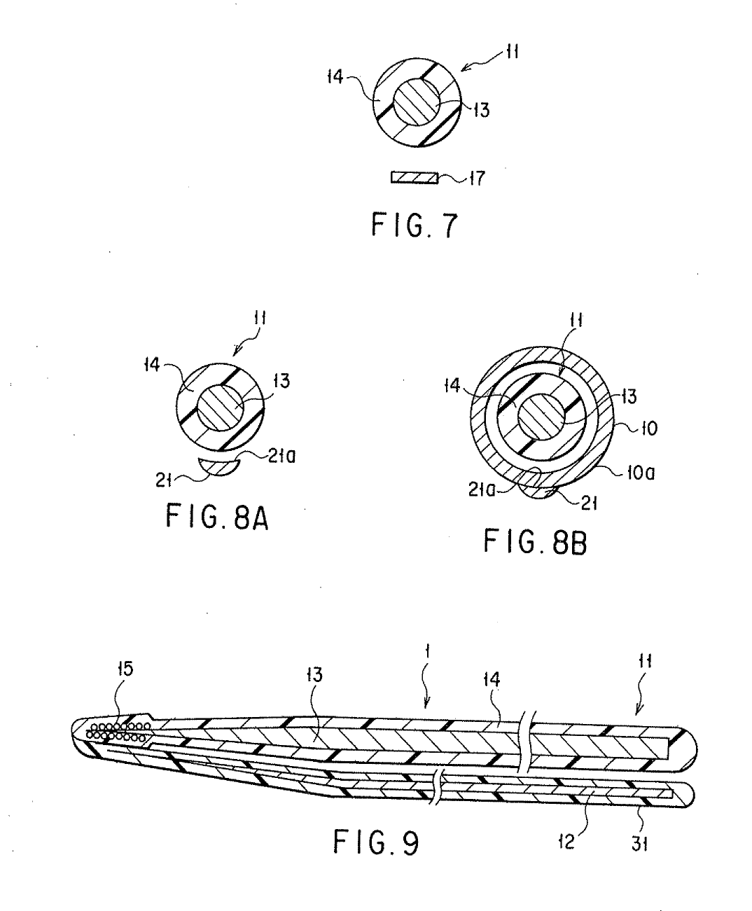

Although the retaining wire 12 has a circular sectional shape according to the present embodiment, as shown in FIG. 3B, moreover, it is not limited to this shape. As in the modification shown in FIG. 7, for example, the medical guide wire 1 may be provided with a ribbon-shaped retaining wire 17 that has a substantially flat sectional shape.

FIGS. 8A and 8B show a second embodiment of the present invention. According to the present embodiment, the configuration of the medical guide wire 1 of the first embodiment (see FIGS. 1 to 6) is modified in the following manner.

More specifically, according to the present embodiment, an arcuate retaining wire 21 having a substantially crescent sectional shape is provided as the retaining wire 12 of the medical guide wire 1, as shown in FIG. 8A. As shown in FIG. 8B, the arcuate shape of the retaining wire 21 is adjusted to the arcuate shape of the outer peripheral surface of the catheter 10 or some other endoscopic appliance that is guided by means of a guide wire body 11.

In working the medical guide wire 1 of the present embodiment, an arcuate surface 21a of the retaining wire 21 is bonded and attached to an outer peripheral surface 10a of the catheter 10 or some other endoscopic appliance so as to extend along the arcuate shape of the surface 10a when the guide wire body 11 is inserted into the tube bore of the catheter 10 or some other endoscopic appliance, as shown in FIG. 8B.

According to the present embodiment, the arcuate retaining wire 21 is provided having a substantially crescent sectional shape. When the guide wire body 11 is attached in a manner such that it is inserted in the tube bore of the catheter 10 or some other endoscopic appliance, therefore, the arcuate surface 21a of the retaining wire 21 can be bonded abutting against the outer peripheral surface 10a of the catheter 10 or some other endoscopic appliance so as to fit its arcuate shape. Accordingly, irregularities on the outer surface side of the catheter 10 or some other endoscopic appliance can be lessened, so that the resistance of insertion of the catheter 10 or some other endoscopic appliance into the appliance passage channel of the endoscope 2 can be reduced. Thus, the ease of insertion of the catheter 10 or some other endoscopic appliance can be improved.

FIG. 9 shows a third embodiment of the present invention. According to the present embodiment, the configuration of the medical guide wire 1 of the first embodiment (see FIGS. 1 to 6) is modified in the following manner.

More specifically, according to the present embodiment, an insulating coating layer 31 is provided around a retaining wire 12. The coating layer 31 of the retaining wire 12 of the present embodiment, like the coating layer 14 around core 13 of a guide wire body 11, is formed of a plastic material, such as fluoroplastic or polyurethane.

The configuration described above has the following effects. According to the present embodiment, the coating layer 31 of the insulator is provided around the retaining wire 12, so that the whole guide wire 1 can be entirely subjected to insulating coating, with the coating layer 14 around the core 13 of the guide wire body 11 and the coating layer 31 around the retaining wire 12. Thus, the operator can be prevented from getting an electric shock or the like if he/she uses a high-frequency appliance, such as a papillotomy knife for excising a papilla.

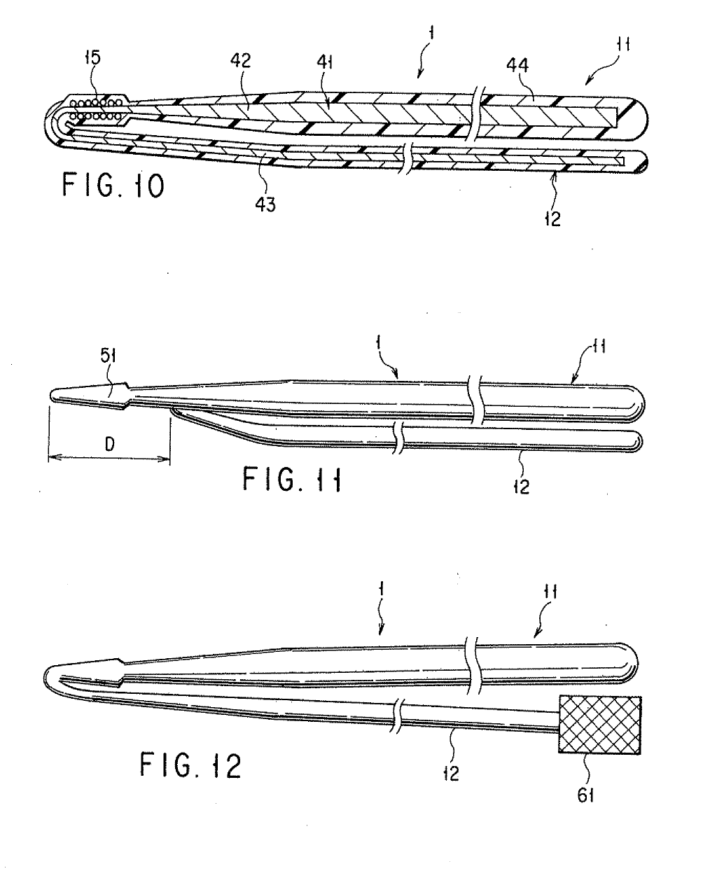

FIG. 10 shows a fourth embodiment of the present invention. According to the present embodiment, the configuration of the medical guide wire 1 of the first embodiment (see FIGS. 1 to 6) is modified in the following manner.

More specifically, according to the present embodiment, one wire 41 is doubled substantially in its central portion so that a guide wire body 11 and a retaining wire 12 are formed on its one fold portion 42 and other fold portion 43, respectively. An insulating coating layer 44 is provided around the whole wire 41 of the present embodiment.

In the configuration described above, the coating layer 44 of the insulator is provided around the whole wire 41 that constitutes the medical guide wire 1, so that the whole guide wire 1 can be insulated. As in the case of the third embodiment (see FIG. 9), therefore, the operator can be prevented from getting an electric shock or the like if he/she uses a high-frequency appliance, such as a papillotomy knife for excising a papilla.

According to the present embodiment, moreover, the one wire 41 is doubled substantially in its central portion so that the guide wire body 11 and the retaining wire 12 are formed on its one fold portion 42 and other fold portion 43, respectively. In the manufacture of the medical guide wire 1, therefore, the operation for bonding the guide wire body 11 and the retaining wire 12 can be omitted, so that manufacturing processes can be simplified, and therefore, costs can be lowered.

FIG. 11 shows a fifth embodiment of the present invention. According to the present embodiment, the configuration of the medical guide wire 1 of the first embodiment (see FIGS. 1 to 6) is modified in the following manner.

More specifically, according to the present embodiment, the distal end portion of a retaining wire 12 is fixed in a position that is situated behind and at a suitable set distance D from the distal end position of a guide wire body 11 of the medical guide wire 1. This set distance D is adjusted to, for example, about 20 to 30 mm. A thin, soft distal portion 51 of the guide wire body 11 alone is formed in a region that covers the suitable set distance D from the distal end position of the guide wire body 11.

According to the present embodiment, the distal end portion of the retaining wire 12 is fixed in the position that is situated behind and at a suitable set distance D from the distal end position of the guide wire body 11. Accordingly, the soft distal portion 51 of the guide wire body 11 alone can be located in the region that covers the suitable set distance D from the distal end position of the guide wire body 11. As in the case where the distal end portion of the retaining wire 12 is fixed to the distal end portion of the guide wire body 11, therefore, a portion that, like the junction of the guide wire body 11 and the retaining wire 12, has an increased outside diameter and higher hardness can be prevented from being located on the distal end portion of the guide wire body 11. In consequence, the distal soft portion 51 of the guide wire body 11 can be softly transformed to fit the shape of the interior of the body cavity as the medical guide wire 1 is inserted into the body cavity that is narrow, so that the ease of insertion of the medical guide wire 1 into the narrow body cavity can be enhanced.

FIG. 12 shows a sixth embodiment of the present invention. According to the present embodiment, the configuration of the medical guide wire 1 of the first embodiment (see FIGS. 1 to 6) is modified in the following manner.

More specifically, according to the present embodiment, the proximal end portion of a retaining wire 12 is provided with a retaining portion 61 that has a diameter larger than that of any other portion. The retaining portion 61, of which the diameter is made larger than that of an appliance inlet hole in the appliance inlet portion 9 of the operating section 4 of the endoscope 2, for example, doubles as a stopper that prevents the proximal end portion of the retaining wire 12 from being inserted into the appliance passage channel of the endoscope 2.

Further, the retaining portion 61 is formed of a material such as a metal, rubber, or elastomer, of which the outer peripheral surface is knurled so that it is not slippery and allows the operator to hold it easily in his/her hand.

According to the present embodiment, the proximal end portion of the retaining wire 12 is provided with the retaining portion 61 that has a diameter larger than that of any other portion. Accordingly, the operator can easily hold the proximal end portion of the retaining wire 12 in his/her hand as he/she manually holds the retaining portion 61, so that the usability of the wire can be improved.

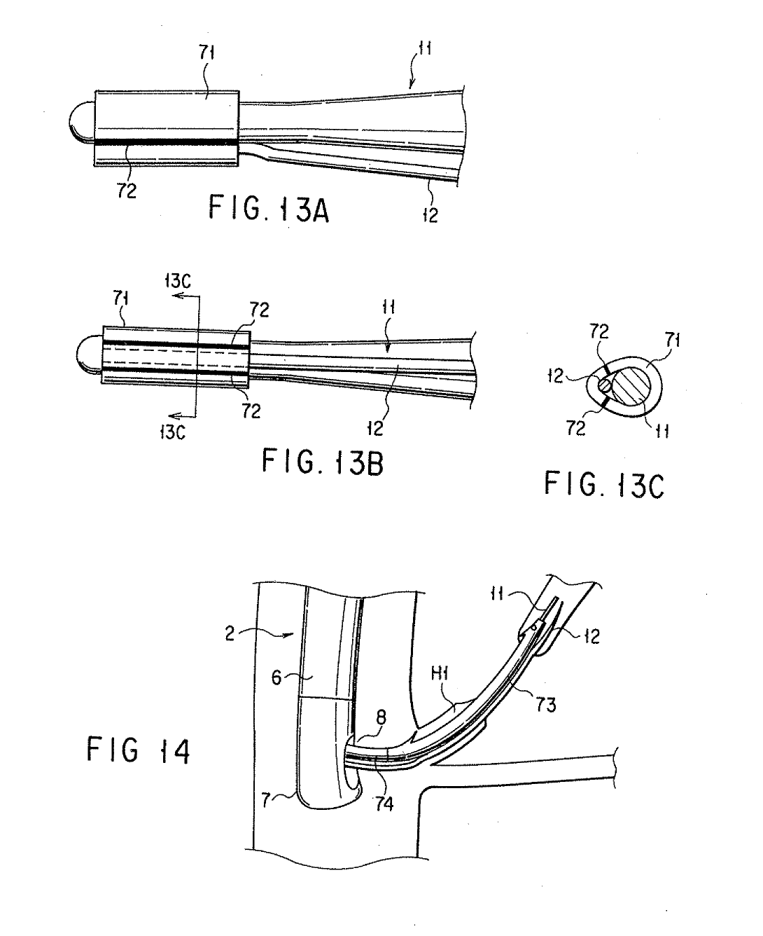

FIGS. 13A to 13C and FIG. 14 show a seventh embodiment of the present invention. According to the present embodiment, the configuration of the medical guide wire 1 of the first embodiment (see FIGS. 1 to 6) is modified in the following manner.

More specifically, according to the present embodiment, a coupling member 71, a soft tube, are provided for coupling the distal end portion of a retaining wire 12 and the distal end portion of a guide wire body 11. Delicate portions 72, which, like peel-away sheaths, for example, are relatively low in strength and easily separable, are provided individually on the opposite sides of a fixing portion of the coupling member 71 for the retaining wire 12. The coupling member 71 is formed of a soft plastic material. Further, the fragile portions 72 are formed of perforations of a perforated heat-shrinkable tube or thermowelded portions, for example.

The following is a description of the function of the configuration described above. In the present embodiment, a drainage tube (stent) 73 to be held in a biliary duct H1 and a pusher tube 74 for pushing in the drainage tube 73 are used as endoscopic appliances, for example, as shown in FIG. 14.

In working the medical guide wire 1 of the present embodiment, moreover, the guide wire body 11 is previously inserted into the respective bores of the drainage tube 73 and the pusher tube 74. As this is done, the drainage tube 73 is set so that it is inserted in a position near the distal end portion of the guide wire body 11.

In this state, the drainage tube 73 and the pusher tube 74, along with the guide wire body 11 of the present embodiment, are inserted into the appliance passage channel through the appliance inlet portion 9 of the operating section 4 of the endoscope 2. The drainage tube 73 and the pusher tube 74 may be inserted in a manner such that the drainage tube 73 is first inserted through the appliance inlet portion 9 and the pusher tube 74 is then inserted, after another endoscopic appliance is drawn out of the medical guide wire 1. As shown in FIG. 14, the drainage tube 73 is caused to project outward from the channel opening 8 of the distal end portion 7 of the insert section 3 and inserted into the biliary duct H1 per papilla.

After the drainage tube 73 is pushed in and moved to an aimed hold position in the biliary duct H1 by means of the pusher tube 74, according to the present embodiment, moreover, the fragile portions 72 of the coupling member 71 are disjoined, and the retaining wire 12 is separated from the guide wire body 11. Thereupon, the guide wire body 11 can be drawn out of the drainage tube 73, so that only the drainage tube 73 can be held in the aimed hold position in the biliary duct H1.

In the configuration described above, the fragile portions 72 are provided individually on the opposite sides of the fixing portion of the coupling member 71, which connects the distal end portion of a retaining wire 12 and the distal end portion of the guide wire body 11, for the retaining wire 12. In inserting the drainage tube 73 into the human body by means of the guide wire 1 and holding it therein, therefore, the retaining wire 12 can be separated from the guide wire body 11, and only the drainage tube 73 can be held in the aimed hold position in the biliary duct H1.

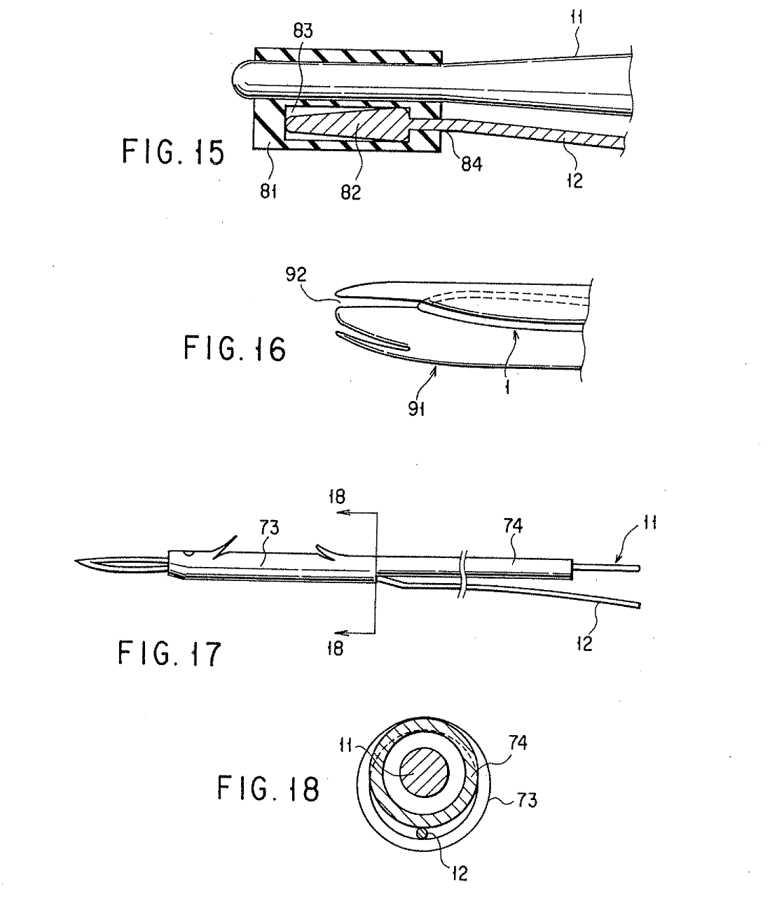

FIG. 15 shows an eighth embodiment of the present invention. According to the present embodiment, the configuration of the medical guide wire 1 of the first embodiment (see FIGS. 1 to 6) is modified in the following manner.

More specifically, according to the present embodiment, a soft coupling member 81 that is formed of an elastic material is provided on the distal end portion of a guide wire body 11, and the distal end portion of a retaining wire 12 is removably coupled to the coupling member 81. A spear-shaped anchor portion 82 is formed on the distal end portion of the retaining wire 12.

Further, the coupling member 81 is formed having a storage chamber 83 for storing the anchor portion 82 of the retaining wire 12 and a slit-shaped plug-in portion 84 located on the rear end portion side of the storage chamber 83. The anchor portion 82 of the retaining wire 12 can be detachably anchored in a manner such that it is inserted into the storage chamber 83 through the plug-in portion 84 of the coupling member 81.

In the configuration described above, the soft coupling member 81 is provided on the distal end portion of a guide wire body 11, and the distal end portion of the retaining wire 12 is removably coupled to the coupling member 81. As in the case of the seventh embodiment (see FIGS. 13A to 13C and FIG. 14), therefore, the retaining wire 12 can be separated from the guide wire body 11, and only the drainage tube 73 can be held in the aimed hold position in the biliary duct H1, in inserting the drainage tube 73 into the human body by means of the guide wire 1 and holding it therein.

According to the present embodiment, moreover, the anchor portion 82 of the retaining wire 12 can be detachably anchored in a manner such that it is inserted into the storage chamber 83 through the plug-in portion 84 of the coupling member 81. Therefore, the retaining wire 12 separated from the guide wire body 11 can be anchored in a manner such that the anchor portion 82 of the retaining wire 12 is inserted again into the storage chamber 83 through the plug-in portion 84 of the coupling member 81. Thus, the retaining wire 12 separated from the guide wire body 11 can be reutilized.

FIG. 16 shows a ninth embodiment of the present invention. According to the present embodiment, an anchor slit 92 to which the medical guide wire 1 is anchored is provided in the distal end portion of an endoscopic appliance 91, such as the catheter 10 according to the first embodiment (see FIGS. 1 to 6) or the drainage tube (stent) 73 according to the seventh embodiment (see FIGS. 13A to 13C and FIG. 14). The junction of the distal end portion of a guide wire body 11 of the medical guide wire 1 and the distal end portion of a retaining wire 12 can be hooked on and detachably anchored to the slit 92. When this is done, the distal end of the medical guide wire 1 is not exposed through the distal end of the endoscopic appliance 91.

An endoscopic appliance that has a slit in its distal end in this manner is disclosed in Jpn. Pat. Appln. KOKAI Publication No. 9-99089, and has conventionally been used in general.

In inserting endoscopic appliances into the human body with the medical guide wire 1 according to the first embodiment used as a guide, the endoscopic appliance to be used first must be inserted together with the medical guide wire that is set in the endoscopic appliance. In the configuration described above, the endoscopic appliance 91 and the medical guide wire 1 can be fixed so as to be immovable relative to each other, by catching and detachably anchoring the junction of the distal end portion of the guide wire body 11 of the medical guide wire 1 and the distal end portion of the retaining wire 12 by means of the 92 of the endoscopic appliance 91. Therefore, the endoscopic appliance 91 and the medical guide wire 1 can be simultaneously inserted with ease.

In inserting the endoscopic appliance to be used first into a papilla, moreover, a technique is generally carried out such that the endoscopic appliance is inserted without using any guide wire in consideration of the ease of insertion into the papilla, and that guide wire is pushed forward after the endoscopic appliance is inserted into the papilla. The foregoing configuration can also cope with this technique.

FIGS. 17 to 19 show a tenth embodiment of the present invention. According to the present embodiment, a drainage tube 73 can be held in the aimed hold position in the biliary duct H1 by using the medical guide wire 1 of the first embodiment (see FIGS. 1 to 6).

More specifically, according to the present embodiment, a guide wire body 11 of the medical guide wire 1 and a retaining wire 12 are inserted together into the bore of the drainage tube 73, and only the guide wire body 11 is inserted into the bore of a pusher tube 74.

According to the present embodiment, the pusher tube 74 is guided in movement by means of the guide wire body 11 of the medical guide wire 1, and the drainage tube 73 is pushed in and moved to the aimed hold position in the biliary duct H1 by means of the pusher tube 74. Thereafter, the guide wire body 11 of the medical guide wire 1 and the retaining wire 12 can be drawn out together from the drainage tube 73. Thereupon, only the drainage tube 73 can be held in the aimed hold position in the biliary duct H1, as shown in FIG. 19.

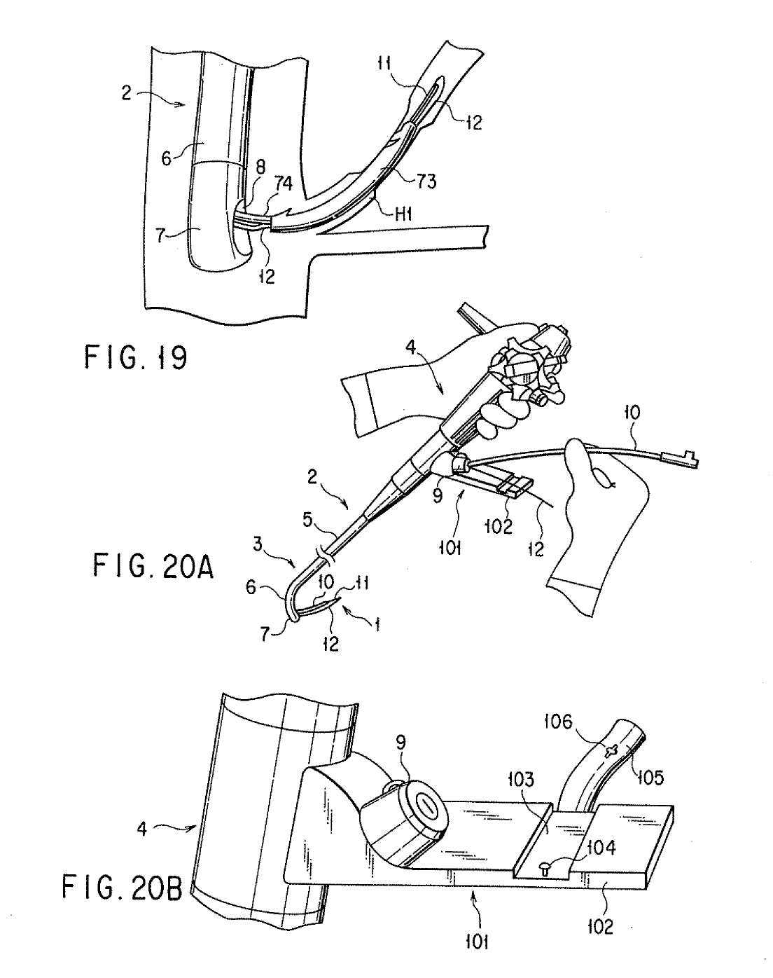

FIGS. 20A and 20B show an eleventh embodiment of the present invention. According to the present embodiment, the appliance inlet portion 9 of the endoscope 2 according to the first embodiment (see FIGS. 1 to 6) is provided with a wire fixture 101 to which the proximal end portion of a retaining wire 12 of a medical guide wire 1, as shown in FIG. 20A.

As shown in FIG. 20B, the wire fixture 101 is provided with a flat wire fixing plate 102. The wire fixing plate 102 is provided with a wire fixing groove 103. Further, an engaging protrusion 104 protrudes from one end portion of the wire fixing groove 103. Furthermore, one end portion of a fixing belt 105 is fixed to the other end portion of the wire fixing groove 103. The other end portion of the fixing belt 105 is formed having a slit-shaped engaging hole portion 106 that detachably engages the engaging protrusion 104.

In working the wire fixture 101, the proximal end portion of the retaining wire 12 of the medical guide wire 1 is located over the wire fixing groove 103 of the wire fixture 101, and the proximal end portion of the retaining wire 12 is pressed against the wire fixing groove 103 by means of the fixing belt 105. In this state, the proximal end portion of the retaining wire 12 of the medical guide wire 1 can be detachably fixed in a manner such that the engaging hole portion 106 of the fixing belt 105 is caused releasably to engage the engaging protrusion 104 of the wire fixing groove 203.

In the configuration described above, the proximal end portion of the retaining wire 12 of the medical guide wire 1 can be detachably fixed with use of the wire fixture 101. As compared with the case where the operator holds the proximal end portion of the retaining wire 12 of the medical guide wire 1 in his/her hand as he/she fixes it, therefore, the operator's operation can be made more labor-saving. Since the operator can insert or remove the endoscopic appliance with the other hand in which he/she does not hold the endoscope, moreover, replacement of the endoscopic appliance can be accomplished without the presence of any assistant at all.

FIG. 21 shows a twelfth embodiment of the present invention. According to the present embodiment, the configuration of the wire fixture 101 of the eleventh embodiment (see FIGS. 20A and 20B) is modified in the following manner.

More specifically, according to the present embodiment, a cylindrical wire fixing mount 111 is provided on a wire fixing plate 102, a wire passage groove through which the proximal end portion of a retaining wire 12 of a medical guide wire 1 is passed is formed on the wire fixing mount 111, and a wire fixing screw 113 is driven into the wire fixing mount 111.

In working the wire fixture 101, the wire fixing screw 113 is driven with the proximal end portion of the retaining wire 12 of the medical guide wire 1 in the wire fixing groove 112, whereupon the proximal end portion of the retaining wire 12 of the medical guide wire 1 is detachably fixed.

According to the present embodiment, the proximal end portion of the retaining wire 12 of the medical guide wire 1 can be also detachably fixed with use of the wire fixture 101. As compared with the case where the operator holds the proximal end portion of the retaining wire 12 of the medical guide wire 1 in his/her hand as he/she fixes it, as in the case of the eleventh embodiment, therefore, the operator's operation can be made more labor-saving. Since the operator can insert or remove the endoscopic appliance with the other hand in which he/she does not hold the endoscope, moreover, replacement of the endoscopic appliance can be accomplished without the presence of any assistant at all.

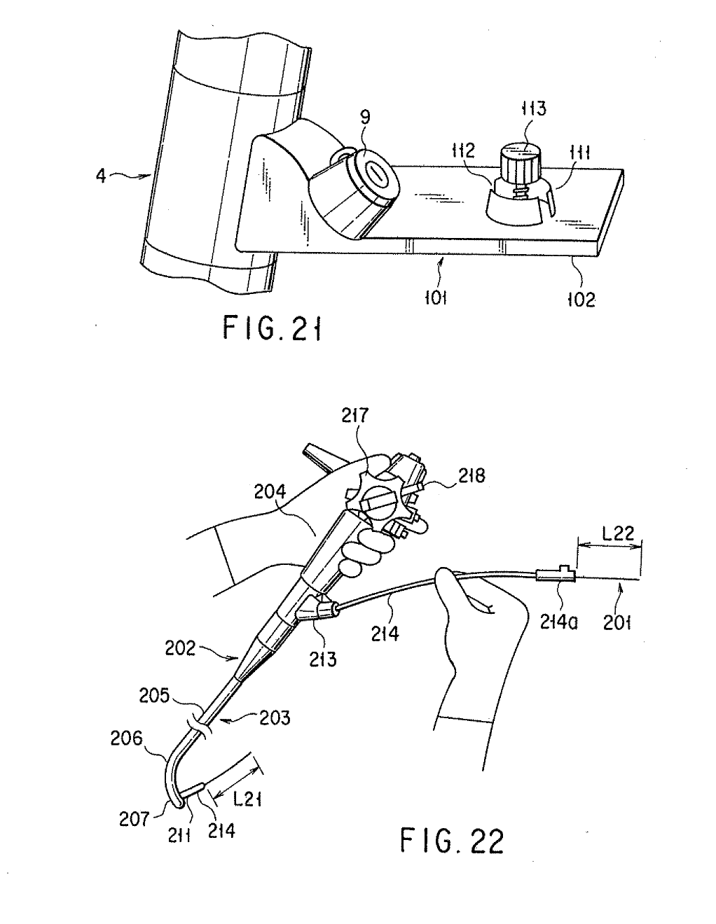

FIGS. 22 to 27 show a thirteenth embodiment of the present invention. FIG. 22 shows the way a medical guide wire 201 of the present embodiment is used in combination with an endoscope 202. The endoscope 202 is provided with an elongate insert section 203 to be inserted into the body cavity, a hand-side operating section 204 coupled to the proximal end portion of the insert section 203, and a universal cord (not shown) to which the proximal end portion of the operating section 204 is coupled. Further, the insert section 203 is provided with components that include an elongate flexible tube portion 205, a curved portion 206 coupled to the distal end of the flexible tube portion 205, and a distal end portion 207 located in the extreme end position of the insert section 203. The endoscope 202 used is the endoscope 202 of a side-vision type for observation in directions substantially perpendicular to the axial direction of the insert section 203.

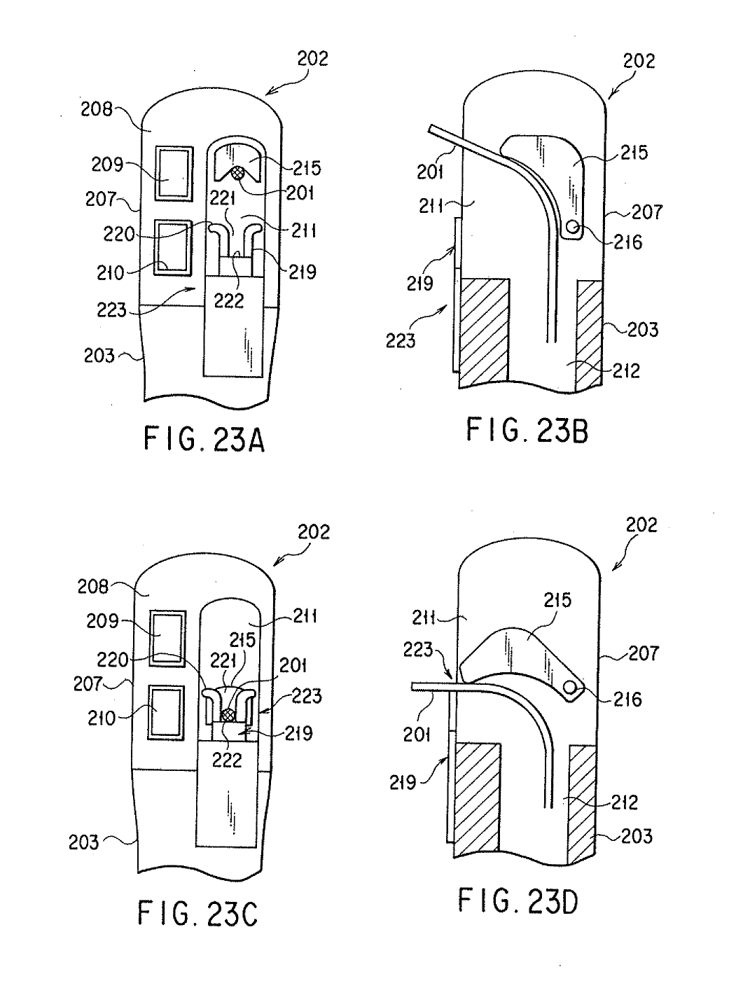

As shown in FIGS. 23A to 23D, the side-vision endoscope 202 is formed having a substantially flat side-vision reference surface 208 that is formed by notching the outer peripheral surface of the distal end portion 207 of the insert section 203. A lighting window 209 of a lighting optical system and an observation window 210 of an observation optical system are juxtaposed in the longitudinal direction on the side-vision reference surface 208. Further, a forceps outlet 211 is located beside the juxtaposition of the lighting window 209 and the observation window 210 on the side-vision reference surface 208. The forceps outlet 211 constitutes a distal end opening of an appliance passage channel 212 as an appliance passage guide way in the insert section 203 of the endoscope 202.

Further, the hand-side operating section 204 is provided with an appliance inlet portion 213 that communicates with the proximal end portion of the appliance passage channel 212. The medical guide wire 201 of the present embodiment, a catheter 214 such as an existing contrastradiography tube with a guide wire lumen through which the guide wire 201 can be passed, or some other endoscopic appliance is alternatively inserted as required into the appliance passage channel 212 through the appliance inlet portion 213 of the hand-side operating section 204, guided to the side of the distal end portion 207 of the insert section 203 through the appliance passage channel 212, and then caused to project outward from the forceps outlet 211 of the distal end portion 207.

A forceps raising block 215 is located on the forceps outlet 211 of the distal end portion 207 of the endoscope 202. One end portion of the forceps raising block 215 is rockably coupled to the body of the distal end portion 207 by means of a pivot 216.

Furthermore, one end portion of a flexible operating wire (not shown) is fixed to the other end portion of the forceps raising block 215. The other end portion of the operating wire extends toward the operating section 204. The operating section 204 is provided with a bending control knob 217 for bending the curved portion 206 in a desired direction and a forceps raising block operating lever 218 for raising the forceps raising block 215. The operating wire is pulled in association with the operation of the forceps raising block operating lever 218 that is attached to the operating section 204. As the operating wire is operated in this manner, the forceps raising block 215 is rocked around the pivot 216. As this is done, the forceps raising block 215 is rocked from a standby position (fallen position) shown in FIGS. 23A and 23B to a maximally rocked position (raised position) shown in FIGS. 23C and 23D. As the forceps raising block 215 is rocked in this manner, operations for raising and leveling the medical guide wire 201 of the present embodiment that extends outward from the forceps outlet 211 and the catheter 214 or some other endoscopic appliance can be carried out in the field of view of the observation window 210.

A guide wire fixture 219 is attached to the side-vision endoscope 202, in the vicinity of the distal end portion 207 of its insert section 203. A appliance receiving portion 220 is formed on the distal end portion side of the guide wire fixture 219. The appliance receiving portion 220 serves to receive other appliance, as well as the guide wire 201.

Further, a guide wire passage groove 221, which is open on its distal end side and has a width of about 1 mm, is formed in the central part of the appliance receiving portion 220. The guide wire 201 can be passed through the guide wire passage groove 221.

A guide wire fixing portion 222 is located in the termination of the guide wire passage groove 221. Any other appliance, as well as the guide wire 201 can be raised, advanced, or retreated in the conventional manner by operating the forceps raising block 215. Only the guide wire 201 can be inserted into the guide wire passage groove 221 of the guide wire fixture 219 by operating the forceps raising block 215, and can be fixed in a manner such that it is anchored between the forceps raising block 215 and the guide wire fixing portion 222 in the termination position of the guide wire passage groove 221. Thereupon, a guide wire fixing mechanism 223 is formed such that the distal end portion of the guide wire 201 is held and detachably anchored between the forceps raising block 215 and the guide wire fixture 219.

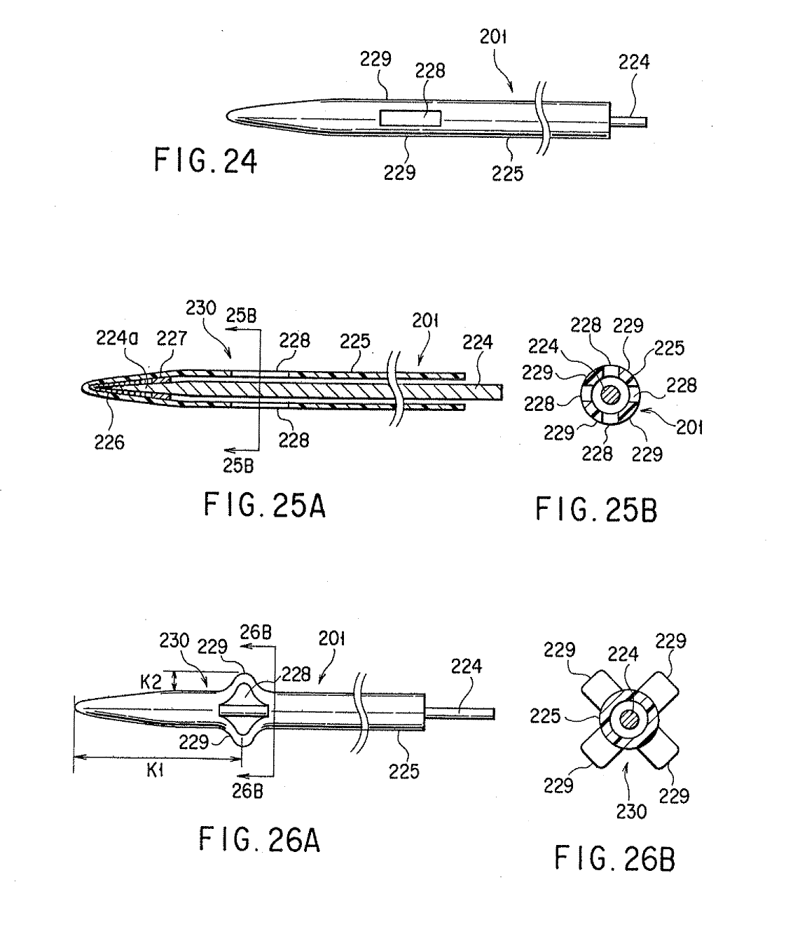

FIG. 24 shows the medical guide wire 201 of the present embodiment. As shown in FIGS. 25A and 25B, the medical guide wire 201 of the present embodiment is provided with an elongate core 224 located in the axial portion of the guide wire 201 and a guide wire sheath 225 that surrounds the core 224.

A tapered portion 224a is formed on the distal end portion of the core 224. Further, an X-ray marker 226 is attached to the distal end portion of the core 224. The X-ray marker 226 is formed by tightly winding a wire of an X-ray-nonpermeable material, such as platinum, gold, silver, palladium, tantalum, or tungsten that does not transmit X-rays, around the tapered portion 224a of the core 224.

The guide wire sheath 225 is formed of a plastic material such as fluoroplastic, polyethylene, or polyurethane. The distal end portion of the guide wire sheath 225 is fixedly bonded to the distal end portion of the core 224 by means of a connecting portion 227 such as an adhesive agent.

On the distal end portion of the guide wire sheath 225, moreover, a plurality of axially elongate slot-shaped slits 22S, four in number, according to the present embodiment, are arranged at equal intervals in the circumferential direction behind the connecting portion 227, as shown in FIG. 25B. Elastically deformable belt-shaped portions 229 are formed between the slits 228.

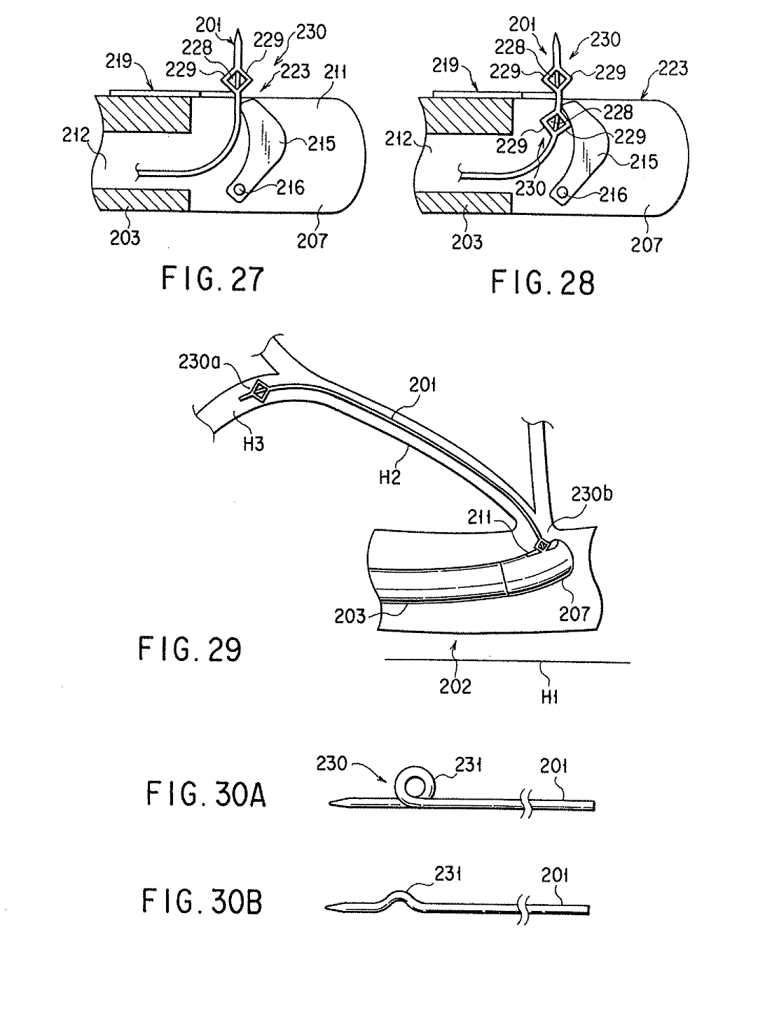

The respective inner peripheral surfaces of the four belt-shaped portions 229 between the slits 228 are held without being bonded to the core 224. As the proximal end portion of the core 224 is pulled to the hand side with respect to the guide wire sheath 225, therefore, the four belt-shaped portions 229 between the slits 228 bulge outward and spread substantially in the shape of a mushroom, as shown in FIGS. 26A and 26B. When the distal end portion of the body of the guide wire 201 is held and detachably anchored by means of the guide wire fixing mechanism 223, in the present embodiment, as shown in FIG. 27, the four belt-shaped portions 229 between the slits 228 of the guide wire sheath 225 are spread substantially in the shape of a mushroom. Thus, an engagement aiding portion 230 is formed that causes the four belt-shaped portions 229, spread substantially in the shape of a mushroom, to engage the guide wire fixing mechanism 223 of the endoscope 202 in a releasable manner, thereby aiding the engagement with the guide wire fixing mechanism 223.

A distance K1 between the distal end of the guide wire 201 and the center position of the engagement aiding portion 230 established when for the four belt-shaped portions 229 are spread substantially in the shape of a mushroom is adjusted to about 20 to 200 mm, for example. Further, a length K2 of an extended portion of each of the four belt-shaped portions 229 of the engagement aiding portion 230 that extends outward from the outer peripheral surface of the guide wire sheath 225 is adjusted to about 1 mm.

The core 224 of the guide wire 201 is not limited to a single wire, and may be formed of a stranded wire or a closely-wound coil. Further, the length of the core 224 of the guide wire 201 is adjusted to about 2,300, for example, and the wire diameter to about 0.9 mm, for example.

The following is a description of the function of the configuration described above. First, in the case where the catheter 214 or some other endoscopic appliance is inserted into the appliance passage channel 212 on the side of the operating section 204 of the endoscope 202, in working the endoscope 202, the distal end portion of the catheter 214 is caused to project from the forceps outlet 211 of the distal end portion 207 of the insert section 203 of the endoscope 202. If the forceps raising block 215 is held in the standby position (fallen position) shown in FIGS. 23A and 23B at this time, the distal end portion of the catheter 214 is held in a position where it is freely movable.

In this state, the forceps raising block operating lever 218 of the operating section 204 of the endoscope 202 is operated, whereupon the forceps raising block 215 of the distal end portion 207 of the insert section 203 is raised. As this operation is performed, the distal end portion of the catheter 214 is pushed out in the direction to raise the forceps by means of the forceps raising block 215, and the raising operation for the catheter 214 is carried out in a regular manner.

With the distal end portion of the guide wire 201 led out of the forceps outlet 211 of the endoscope 202, as shown in FIGS. 23A and 23B, the forceps raising block operating lever 218 of the operating section 204 of the endoscope 202 is operated to raise the forceps raising block 215. As the forceps raising block 215 is raised, in this case, the guide wire 201 is inserted into the guide wire passage groove 221 of the appliance receiving portion 220 of the guide wire fixture 219. When the forceps raising block 215 is rocked to the maximally rocked position (raised position) shown in FIGS. 23C and 23D, the guide wire 201 is pressed against the guide wire fixing portion 222 of the guide wire fixture 219 by means of a push force from the forceps raising block 215 in the termination position of the guide wire passage groove 221. As this is done, the guide wire 201 is fixed in a manner such that it is anchored between the forceps raising block 215 and the guide wire fixing portion 222.

When the distal end portion of the body of the guide wire 201 is held and detachably anchored by means of the guide wire fixing mechanism 223, according to the present embodiment, moreover, the proximal end portion of the core 224 is pulled to the hand side with respect to the guide wire sheath 225. By doing this, the four belt-shaped portions 229 between the slits 228 of the guide wire sheath 225 are spread substantially in the shape of a mushroom, thereby forming the engagement aiding portion 230, as shown in FIG. 27. Thereupon, the four belt-shaped portions 229 spread substantially in the shape of a mushroom are caused releasably to engage the guide wire fixing mechanism 223 of the endoscope 202, whereby the engagement with the guide wire fixing mechanism 223 is aided.

After the catheter 214 or some other endoscopic appliance is inserted into the pancreatic or biliary duct (not shown) per papilla, in working the endoscope 202, the catheter 214 is replaced in the following manner. First, the guide wire 201 of the present embodiment is inserted through a mouthpiece 214a on the proximal end side of the catheter 214 and introduced into the pancreatic or biliary duct (not shown). In this case, a stretch (length) L21 of the distal end portion of the guide wire 201 that projects from the distal end of the catheter 214 is adjusted to about 20 to 200 mm, for example, and a stretch (length) L22 of the proximal end portion of the guide wire 201 that projects from the mouthpiece 214a on the proximal end side of the catheter 214 is adjusted to about 5 to 200 mm, for example.

The introduction of the guide wire 201 into the pancreatic or biliary duct (not shown) is confirmed, and the catheter 214 is drawn out in a manner such that the proximal end side of the guide wire 201 is manually held, to prevent the guide wire 201 from moving. As this is done, the catheter 214 is further drawn out after it is confirmed that the distal end portion of the catheter 214 is drawn out of the papilla (not shown).

When the distal end of the catheter 214 is then set in the forceps outlet 211 on the side of the distal end portion 207 of the insert section 203 of the endoscope 202, the guide wire 201 is mechanically fixed near the distal end portion 207 of the insert section 203 of the endoscope 202 by means of the guide wire fixing mechanism 223. As this is done, the four belt-shaped portions 229 at the distal end portion of the guide wire sheath 225 of the guide wire 201 are spread substantially in the shape of a mushroom to form the engagement aiding portion 230. As the engagement aiding portion 230 is caused releasably to engage the guide wire fixing mechanism 223 of the endoscope 202, the engagement with the guide wire fixing mechanism 223 is aided.

After it is confirmed that the guide wire 201 is fixed, moreover, the catheter 214 is entirely drawn out of the operating section 204 of the endoscope 202. Thereafter, an appliance to be used next is inserted from the proximal end side of the guide wire 201 and inserted into the pancreatic or biliary duct (not shown) with the guide wire 201 used as a guide.

In replacing the catheter 214, therefore, the operator need not hold the guide wire 201. Thereafter, the catheter 214 or some other endoscopic appliance can be replaced as needed by the same method.

The configuration described above has the following effects. In the medical guide wire 201 of the present embodiment, the distal end portion of the body of the guide wire 201 is provided with the engagement aiding portion 230 for aiding the engagement with the guide wire fixing mechanism 223 on the side of the endoscope 202. When the distal end portion of the body of the guide wire 201 is held and detachably anchored by means of the guide wire fixing mechanism 223 on the side of the endoscope 202, therefore, the four belt-shaped portions 229 between the slits 228 of the guide wire sheath 225 are spread substantially in the shape of a mushroom. By doing this, the four belt-shaped portions 229 that are spread substantially in the shape of a mushroom can be caused releasably to engage the guide wire fixing mechanism 223 of the endoscope 202, thereby aiding the engagement with the guide wire fixing mechanism 223. In replacing the catheter 214 or some other endoscopic appliance, therefore, the guide wire 201 can be securely anchored between the forceps raising block 215 and the guide wire fixing portion 222 of the guide wire fixture 219 by operating the forceps raising block 215. In contrast with the conventional case, therefore, the necessity of holding the guide wire 201 on the side of the operating section 204 of the endoscope 202 can be obviated, and the length of the guide wire 201 itself can be adjusted to a length of about 2,300 mm. Thus, the guide wire 201 itself can be made shorter than a conventional one, and the operation for replacing the endoscopic appliance can be facilitated, so that the required operating time for the appliance replacement operation can be shortened. Since the configuration on the appliance side need not be changed at all, moreover, a conventional appliance can be used, and the appliance replacement operation can be easily carried out without interfering with the conventional operating method or the sense of operation.

Although the engagement aiding portion 230 that is spread substantially in the shape of a mushroom is provided in one position on the distal end portion of the guide wire sheath 225, as shown in FIG. 27, according to the present embodiment, the invention is not limited to this arrangement. As in the case of a first modification of the medical guide wire 201 shown in FIG. 28, for example, two engagement aiding portions 230 may be arranged in the axial direction on the distal end portion of the guide wire sheath 225. In this case, the guide wire 201 can be caused releasably to engage the guide wire fixing mechanism 223 of the endoscope 202 to aid the engagement with the guide wire fixing mechanism 223, not only in a direction such that the guide wire 201 is drawn out to the hand side but also in a direction such that the guide wire 201 is inserted in.

As in the case of a second modification of the medical guide wire 201 shown in FIG. 29, moreover, a first engagement aiding portion 230a and a second engagement aiding portion 230b may be provided, respectively, on the distal end portion of the guide wire 201 and in a position behind the first engagement aiding portion 230a, e.g., in a position at a distance of, for example, 130 mm or more. In this case, the guide wire 201 that is caused project outward form the forceps outlet 211 may be inserted into a hepatic duct H3 through a common bile duct H2 with the distal end portion 207 of the endoscope 202 in a duodenum H1, for example. When this is done, the second engagement aiding portion 230b can be caused releasably to engage the guide wire fixing mechanism 223 of the endoscope 202, thereby aiding the engagement with the guide wire fixing mechanism 223, and the first engagement aiding portion 230a of the distal end portion of the guide wire 201 can be anchored in the hepatic duct H3. With this configuration, the guide wire 201 can be fixed both in a position where the guide wire 201 is located in the hepatic duct H3 and in a position in the common bile duct H2 at a short distance from the duodenal papilla.

FIGS. 30A, 30B and 31 show a fourteenth embodiment of the present invention. According to the present embodiment, the configuration of the medical guide wire 201 of the thirteenth embodiment (see FIGS. 22 to 27) is modified in the following manner.

More specifically, according to the present embodiment, a crooked preshaped portion 231 is provided as the engagement aiding portion 230 of the medical guide wire 201 on the distal end portion of the guide wire 201, as shown in FIGS. 30A and 30B. When the guide wire 201 is inserted into the tube bore of the catheter 214 or some other endoscopic appliance, moreover, the preshaped portion 231 is inserted into the tube bore of the catheter 214 or some other endoscopic appliance in a manner such that it is elastically deformed in a substantially straight stretched shape. A plurality of preshaped portions 231 may be arranged in the axial direction of the guide wire 201.

In working the medical guide wire 201 of the present embodiment, the preshaped portion 231 on the distal end portion of the guide wire 201 can be caused releasably to engage the guide wire fixing mechanism 223 of the endoscope 202, thereby aiding the engagement with the guide wire fixing mechanism 223, as shown in FIG. 31.

According to the present embodiment, the crooked preshaped portion 231 is provided on the distal end portion of the guide wire 201, and this preshaped portion 231 is caused releasably to engage the guide wire fixing mechanism 223 of the endoscope 202, thereby aiding the engagement with the guide wire fixing mechanism 223. According to the present embodiment, therefore, the guide wire 201 can be also securely fixed by means of the guide wire fixing mechanism 223, so that the same effects of the thirteenth embodiment can be obtained.

FIGS. 32 and 33 show a fifteenth embodiment of the present invention. According to the present embodiment, the configuration of the medical guide wire 201 of the thirteenth embodiment (see FIGS. 22 to 27) is modified in the following manner.

More specifically, according to the present embodiment, a small-diameter portion 241 having an outside diameter smaller than that of any other portion is provided as the engagement aiding portion 230 of the medical guide wire 201 on the distal end portion of the guide wire 201, as shown in FIG. 32. The depth of grooves of the small-diameter portion 241 are adjusted to about 0.1 to 0.3 mm. Further, a distance L3 between the front end portion of the small-diameter portion 241 and the distal end position of the guide wire 201 is adjusted to about 20 to 30 mm, and a distance L4 between the rear end portion of the small-diameter portion 241 and the distal end position of the guide wire 201 to about 150 to 200 mm.

In working the medical guide wire 201 of the present embodiment, a stepped portion of the small-diameter portion 241 on the distal end portion of the guide wire 201 can be caused releasably to engage the guide wire fixing mechanism 223 of the endoscope 202, thereby aiding the engagement with the guide wire fixing mechanism 223, as shown in FIG. 33.

According to the present embodiment, the small-diameter portion 241 having an outside diameter smaller than that of any other portion is provided on the distal end portion of the guide wire 201, and the stepped portion of the small-diameter portion 241 is caused releasably to engage the guide wire fixing mechanism 223 of the endoscope 202, thereby aiding the engagement with the guide wire fixing mechanism 223. Therefore, the guide wire fixing mechanism 223 of the endoscope 202 catches the stepped portion of the small-diameter portion 241 of the guide wire 201, thereby preventing the guide wire 201 from advancing or retreating beyond this point. According to the present embodiment, therefore, the guide wire 201 can be also securely fixed by means of the guide wire fixing mechanism 223, so that the same effects of the thirteenth embodiment can be obtained.

According to the present embodiment, moreover, the small-diameter portion 241 is simply provided as the engagement aiding portion 230 of the medical guide wire 201 on the distal end portion of the guide wire 201, as shown in FIG. 32, so that manufacture is easy. According to the present embodiment, furthermore, there is an effect such that the guide wire 201 can be securely fixed in the direction of its insertion as well as in the direction of its removal by means of the one small-diameter portion 241 alone.

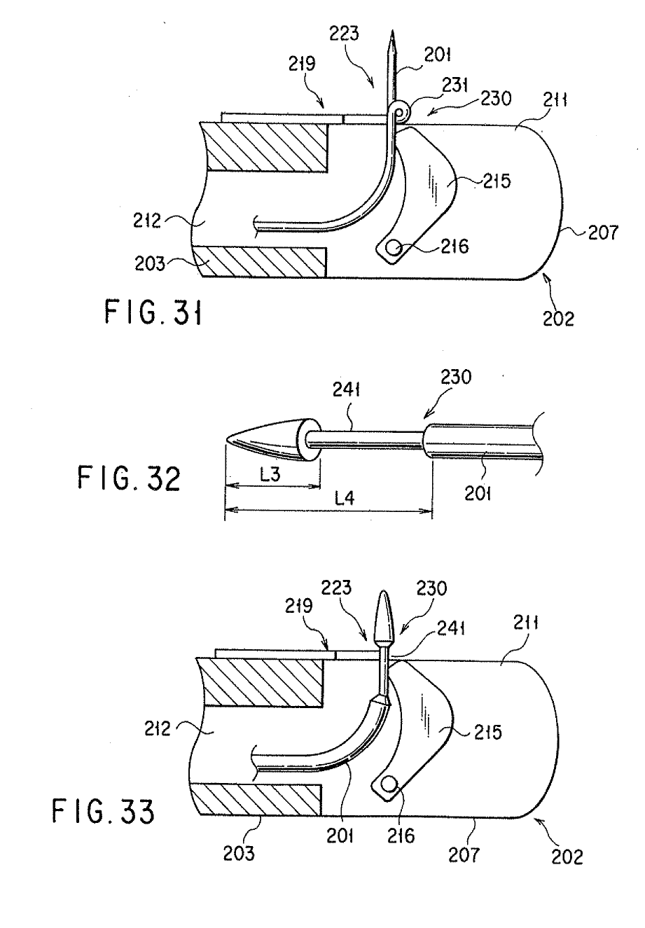

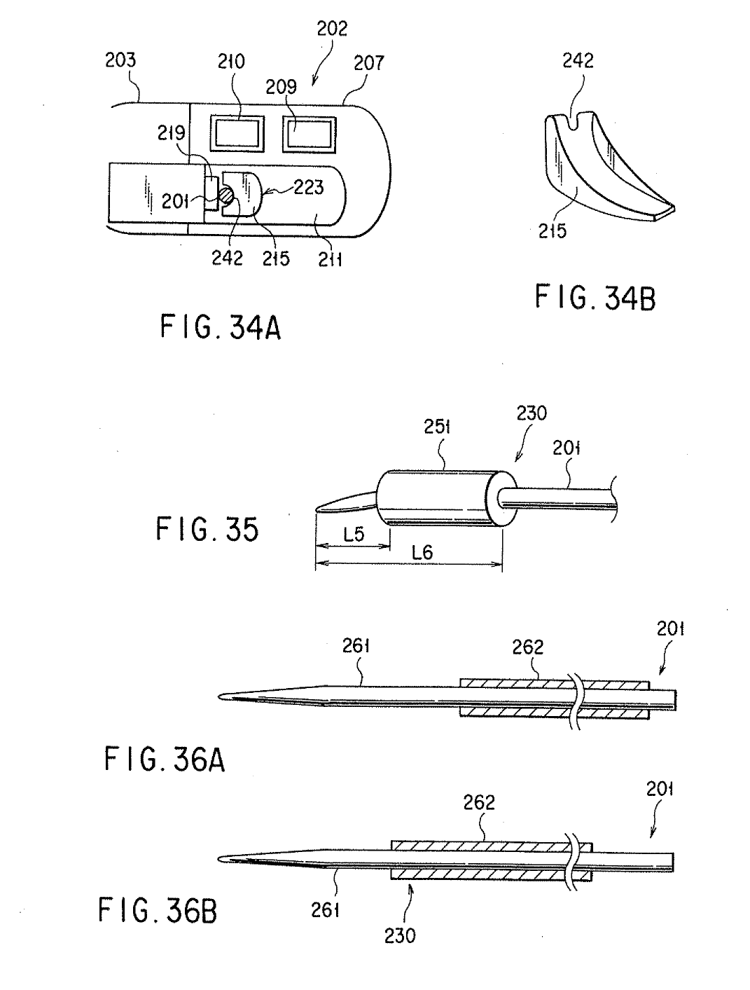

FIGS. 34A and 34B show a sixteenth embodiment of the present invention. According to the present embodiment, the forceps raising block 215 of the endoscope 202 that receives the medical guide wire 201 of the fifteenth embodiment (see FIGS. 32 and 33) is provided with an engaging groove 242 having a size that matches the small-diameter portion 241 of the guide wire 201.

In working the medical guide wire 201 of the present embodiment, the engagement with the guide wire fixing mechanism 223 of the endoscope 202 can be aided as the small-diameter portion 241 of the guide wire 201 engages the engaging groove 242 of the forceps raising block 215 of the endoscope 202 when the stepped portion of the small-diameter portion 241 on the distal end portion of the guide wire 201 is caused releasably to engage the guide wire fixing mechanism 223.

FIG. 35 shows a seventeenth embodiment of the present invention. According to the present embodiment, the configuration of the medical guide wire 201 of the thirteenth embodiment (see FIGS. 22 to 27) is modified in the following manner.

More specifically, according to the present embodiment, a large-diameter portion 251 having an outside diameter larger than that of any other portion is provided as the engagement aiding portion 230 of the medical guide wire 201 on the distal end portion of the guide wire 201, as shown in FIG. 35. The height of the large-diameter portion 251 is adjusted to about 0.1 to 0.3 mm. Further, a distance L5 between the front end portion of the large-diameter portion 251 and the distal end position of the guide wire 201 is adjusted to about 20 to 30 mm, and a distance L6 between the rear end portion of the large-diameter portion 251 and the distal end position of the guide wire 201 to about 150 to 200 mm.

In working the medical guide wire 201 of the present embodiment, the large-diameter portion 251 on the distal end portion of the guide wire 201 is caused releasably to engage the guide wire fixing mechanism 223 of the endoscope 202. By doing this, the area of contact between the guide wire fixing mechanism 223 of the endoscope 202 and the guide wire 201 can be increased to enhance fixing force that is based on frictional resistance, thereby aiding the engagement with the guide wire fixing mechanism 223.

According to the present embodiment, the large-diameter portion 251 having an outside diameter larger than that of any other portion is provided on the distal end portion of the guide wire 201. This large-diameter portion 251 is caused releasably to engage the guide wire fixing mechanism 223 of the endoscope 202, whereby the area of contact between the guide wire fixing mechanism 223 of the endoscope 202 and the guide wire 201 can be increased to enhance the fixing force that is based on the frictional resistance, so that the engagement with the guide wire fixing mechanism 223 can be aided. According to the present embodiment, therefore, the guide wire 201 can be also securely fixed by means of the guide wire fixing mechanism 223, so that the same effects of the thirteenth embodiment can be obtained.

FIGS. 36A and 36B show an eighteenth embodiment of the present invention. According to the present embodiment, the configuration of the medical guide wire 201 of the thirteenth embodiment (see FIGS. 22 to 27) is modified in the following manner.

More specifically, according to the present embodiment, the medical guide wire 201 of the present embodiment is provided with an elongate core 261 located in the axial portion of the guide wire 201 and a guide wire sheath 262 that is slidable in the axial direction along the core 261, as shown in FIGS. 36A and 36B. The guide wire sheath 262 is supported so as to be slidable from a standby position (retreated position) shown in FIG. 36A to an advanced position shown in FIG. 36B.

In working the medical guide wire 201 of the present embodiment, the guide wire sheath 262 is slid to the advanced position shown in FIG. 36B, whereby a large-diameter portion having an outside diameter larger than that of the core 261 can be provided on the distal end portion of the guide wire 201, as in the case of the seventeenth embodiment (see FIG. 35). In this state, the large-diameter portion based on the guide wire sheath 262 is caused releasably to engage the guide wire fixing mechanism 223 of the endoscope 202, whereupon the area of contact between the guide wire fixing mechanism 223 of the endoscope 202 and the guide wire 201 can be increased, to enhance the fixing force that is based on frictional resistance. This structure can be made to function as the engagement aiding portion 230 for aiding the engagement with the guide wire fixing mechanism 223.

According to the present embodiment, moreover, the outside diameter of the distal end portion of the guide wire 201 can be maintained without exceeding the outside diameter of the core 261 alone by retreating the guide wire sheath 262 to the standby position (retreated position) shown in FIG. 36A. Thus, the location of the large-diameter portion on the distal end portion of the guide wire 201 can be prevented from lowering the ease of insertion of the guide wire 201 into the pancreatic duct, biliary duct, or the like.

If necessary, according to the present embodiment, therefore, the guide wire sheath 262 can be made to function as the engagement aiding portion 230 that is slid to the advanced position shown in FIG. 36B to aid the engagement with the guide wire fixing mechanism 223. When it need not be worked, the guide wire sheath 262 can be retreated to the standby position (retreated position) shown in FIG. 36A, thereby preventing lowering of the ease of insertion of the guide wire 201 into the pancreatic duct, biliary duct, or the like.

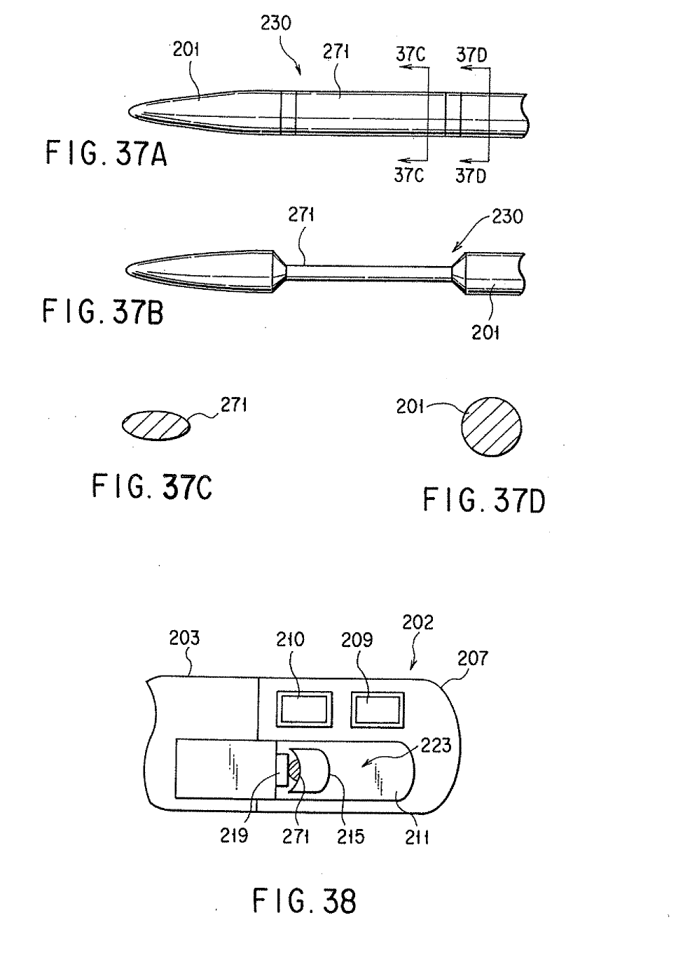

FIGS. 37A to 37D and 38 show a nineteenth embodiment of the present invention. According to the present embodiment, the configuration of the medical guide wire 201 of the thirteenth embodiment (see FIGS. 22 to 27) is modified in the following manner.

More specifically, according to the present embodiment, a flat portion 271 is provided as the engagement aiding portion 230 of the medical guide wire 201 on the distal end portion of the guide wire 201, as shown in FIGS. 37A and 37B. As shown in FIG. 37C, the sectional shape of the flat portion 271 is substantially elliptic. FIG. 37D shows a circular sectional shape of any other portion of the guide wire 201 than the flat portion 271.

In working the medical guide wire 201 of the present embodiment, the flat portion 271 on the distal end portion of the guide wire 201 is caused releasably to engage the guide wire fixing mechanism 223 of the endoscope 202, as shown in FIG. 38, whereupon the area of contact between the guide wire fixing mechanism 223 of the endoscope 202 and the flat portion 271 of the guide wire 201 can be increased to enhance fixing force that is based on frictional resistance. Thus, the engagement with the guide wire fixing mechanism 223 can be aided.

According to the present embodiment, the flat portion 271 is provided on the distal end portion of the guide wire 201, and this flat portion 271 is caused releasably to engage the guide wire fixing mechanism 223 of the endoscope 202. By doing this, the area of contact between the guide wire fixing mechanism 223 of the endoscope 202 and the flat portion 271 of the guide wire 201 can be increased to enhance the fixing force that is based oh the frictional resistance, thereby aiding the engagement with the guide wire fixing mechanism 223. According to the present embodiment, therefore, the guide wire 201 can be also securely fixed by means of the guide wire fixing mechanism 223, so that the same effects of the thirteenth embodiment can be obtained.

According to the present embodiment, furthermore, a stepped portion of the flat portion 271 of the guide wire 201 can be caused releasably to engage the guide wire fixing mechanism 223 of the endoscope 202, thereby aiding the engagement with the guide wire fixing mechanism 223.

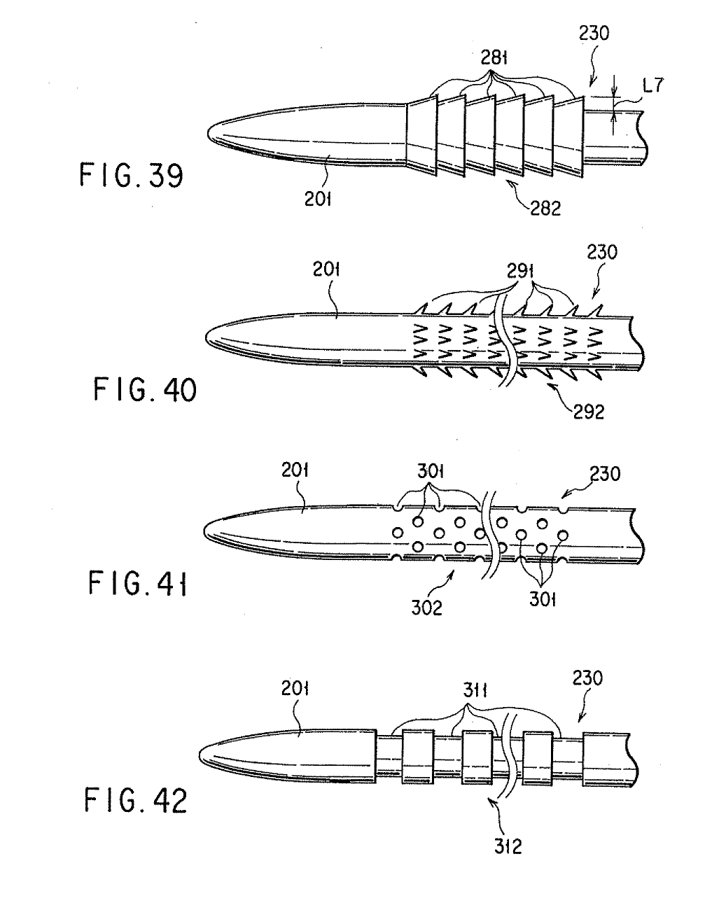

FIG. 39 shows a twentieth embodiment of the present invention. According to the present embodiment, the configuration of the medical guide wire 201 of the thirteenth embodiment (see FIGS. 22 to 27) is modified in the following manner.

More specifically, according to the present embodiment, a substantially serrate rugged portion 282, which includes a plurality of substantially chevron-shaped projections juxtaposed in the axial direction, is provided as the engagement aiding portion 230 of the medical guide wire 201 on the distal end portion of the guide wire 201, as shown in FIG. 39. A rise (height) L7 of each projection 281 is adjusted to about 0.1 to 0.3 mm, for example.

In working the medical guide wire 201 of the present embodiment, the rugged portion 282 on the distal end portion of the guide wire 201 can be caused releasably to engage the guide wire fixing mechanism 223 of the endoscope 202 so as to be hooked on it, thereby aiding the engagement between the guide wire 201 and the guide wire fixing mechanism 223 of the endoscope 202.

FIG. 40 shows a twenty-first embodiment of the present invention. According to the present embodiment, the configuration of the medical guide wire 201 of the thirteenth embodiment (see FIGS. 22 to 27) is modified in the following manner.

More specifically, according to the present embodiment, a rugged portion 292, which includes a large number of projecting spines 291, is provided as the engagement aiding portion 230 of the medical guide wire 201 on the distal end portion of the guide wire 201, as shown in FIG. 40.

In working the medical guide wire 201 of the present embodiment, the rugged portion 292 on the distal end portion of the guide wire 201 can be caused releasably to engage the guide wire fixing mechanism 223 of the endoscope 202, thereby preventing the guide wire 201 from easily slipping off and aiding the engagement between the guide wire 201 and the guide wire fixing mechanism 223 of the endoscope 202.

FIG. 41 shows a twenty-second embodiment of the present invention. According to the present embodiment, the configuration of the medical guide wire 201 of the thirteenth embodiment (see FIGS. 22 to 27) is modified in the following manner.

More specifically, according to the present embodiment, a dimpled portion 302, which includes a large number of dimples 301 formed by laser working, is provided as the engagement aiding portion 230 of the medical guide wire 201 on the distal end portion of the guide wire 201, as shown in FIG. 41.

In working the medical guide wire 201 of the present embodiment, the dimpled portion 302 on the distal end portion of the guide wire 201 can be caused releasably to engage the guide wire fixing mechanism 223 of the endoscope 202, thereby preventing the guide wire 201 from easily moving back and forth and aiding the engagement between the guide wire 201 and the guide wire fixing mechanism 223 of the endoscope 202.

FIG. 42 shows a twenty-third embodiment of the present invention. According to the present embodiment, the configuration of the medical guide wire 201 of the thirteenth embodiment (see FIGS. 22 to 27) is modified in the following manner.

More specifically, according to the present embodiment, a chased portion 312, which includes a plurality of ring-shaped grooves 311 formed by laser working, is provided as the engagement aiding portion 230 of the medical guide wire 201 on the distal end portion of the guide wire 201, as shown in FIG. 42.

In working the medical guide wire 201 of the present embodiment, the chased portion 312 on the distal end portion of the guide wire 201 can be caused releasably to engage the guide wire fixing mechanism 223 of the endoscope 202, thereby preventing the guide wire 201 from easily moving back and forth and aiding the engagement between the guide wire 201 and the guide wire fixing mechanism 223 of the endoscope 202.

Instead of the chased portion 312 that includes a plurality of juxtaposed ring-shaped grooves 311, a chased portion that includes a spiral groove formed by laser working may be provided on the distal end portion of the guide wire 201.

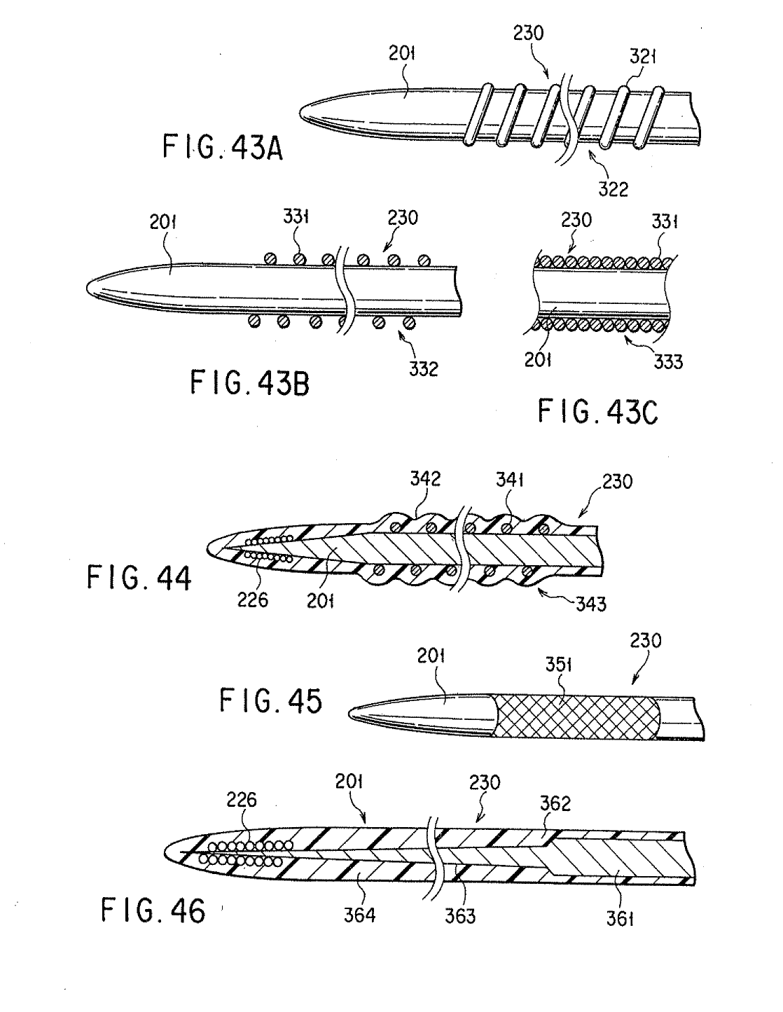

FIG. 43A shows a twenty-fourth embodiment of the present invention. According to the present embodiment, the configuration of the medical guide wire 201 of the thirteenth embodiment (see FIGS. 22 to 27) is modified in the following manner.

More specifically, according to the present embodiment, a rugged portion 322 in the form of a spiral groove 322, which is defined by a projecting spiral ridge 321, is provided as the engagement aiding portion 230 of the medical guide wire 201 on the distal end portion of the guide wire 201, as shown in FIG. 43A.

In working the medical guide wire 201 of the present embodiment, the rugged portion 322 in the form of a spiral groove on the distal end portion of the guide wire 201 can be caused releasably to engage the guide wire fixing mechanism 223 of the endoscope 202 so as to be hooked on it, thereby preventing the guide wire 201 from easily moving back and forth and aiding the engagement between the guide wire 201 and the guide wire fixing mechanism 223 of the endoscope 202.

FIG. 43B shows a first modification of the twenty-fourth embodiment (see FIG. 43A). According to the present modification, a rugged portion 332 that resembles the spiral-groove-shaped rugged portion 322 of the twenty-fourth embodiment is formed by spirally winding a wire element 331, such as a thread or wire with a wire diameter of about 0.1 to 0.3 mm, for example, around the distal end portion of the guide wire 201.

In working the medical guide wire 201 of the present modification, the rugged portion 332 in the form of a spiral groove on the distal end portion of the guide wire 201 can be caused releasably to engage the guide wire fixing mechanism 223 of the endoscope 202 so as to be hooked on it, thereby preventing the guide wire 201 from easily moving back and forth and aiding the engagement between the guide wire 201 and the guide wire fixing mechanism 223 of the endoscope 202.

FIG. 43C shows a second modification of the twenty-fourth embodiment (see FIG. 43A). According to the present modification, a rugged portion 333 in the form of a closely-wound coil is formed by winding the wire element 331 of the first modification at fine pitches like a closely-wound coil around the distal end portion of the guide wire 201. The present modification can produce the same effects of the first modification.

FIG. 44 shows a twenty-fifth embodiment of the present invention. According to the present embodiment, the configuration of the medical guide wire 201 of the thirteenth embodiment (see FIGS. 22 to 27) is modified in the following manner.

More specifically, according to the present embodiment, a rugged portion 343 in the form of a spiral groove is provided as the engagement aiding portion 230 of the medical guide wire 201 by winding a roughly-wound coil 341 around the distal end portion of the guide wire 201 and then coating the whole outer peripheral surface of the guide wire 201 with a coating layer 342 of plastic or the like, as shown in FIG. 44.

In working the medical guide wire 201 of the present embodiment, the rugged portion 343 in the form of a spiral groove on the distal end portion of the guide wire 201 can be caused releasably to engage the guide wire fixing mechanism 223 of the endoscope 202 so as to be hooked on it, thereby preventing the guide wire 201 from easily moving back and forth and aiding the engagement between the guide wire 201 and the guide wire fixing mechanism 223 of the endoscope 202.

FIG. 45 shows a twenty-sixth embodiment of the present invention. According to the present embodiment, the configuration of the medical guide wire 201 of the thirteenth embodiment (see FIGS. 22 to 27) is modified in the following manner.

More specifically, according to the present embodiment, a high-friction portion 351 of an unslippery high-friction material is provided as the engagement aiding portion 230 of the medical guide wire 201 on the distal end portion of the guide wire 201, as shown in FIG. 45. The high-friction portion 351 is formed of rubber, silicone, or any of various elastomers with the Shore A-hardness of about 90 or less, for example.

In working the medical guide wire 201 of the present embodiment, the high-friction portion 351 on the distal end portion of the guide wire 201 can be caused releasably to engage the guide wire fixing mechanism 223 of the endoscope 202 so as to touch it, thereby preventing the guide wire 201 from easily moving back and forth and aiding the engagement between the guide wire 201 and the guide wire fixing mechanism 223 of the endoscope 202. According to the present embodiment, moreover, the guide wire 201 can be further prevented from easily moving back and forth by causing a contact portion on the side of the guide wire fixing mechanism 223 of the endoscope 202 to bite into the high-friction portion 351.

According to the present embodiment, moreover, the guide wire 201 has no difference in level, so that the guide wire 201 can be easily inserted into the body cavity and passed through the appliance. According to the present embodiment, furthermore, the guide wire 201 can be securely fixed in both the directions of insertion and removal when the guide wire 201 is anchored.

A contact member of the same material with the forceps raising block 215 on the side of the guide wire fixing mechanism 223 of the endoscope 202 and a contact portion of the guide wire fixture 219 may be provided as the high-friction portion 351 of the present embodiment. In this case, the guide wire 201 can be also prevented from easily moving back and forth, and the engagement between the guide wire 201 and the guide wire fixing mechanism 223 of the endoscope 202 can be aided.

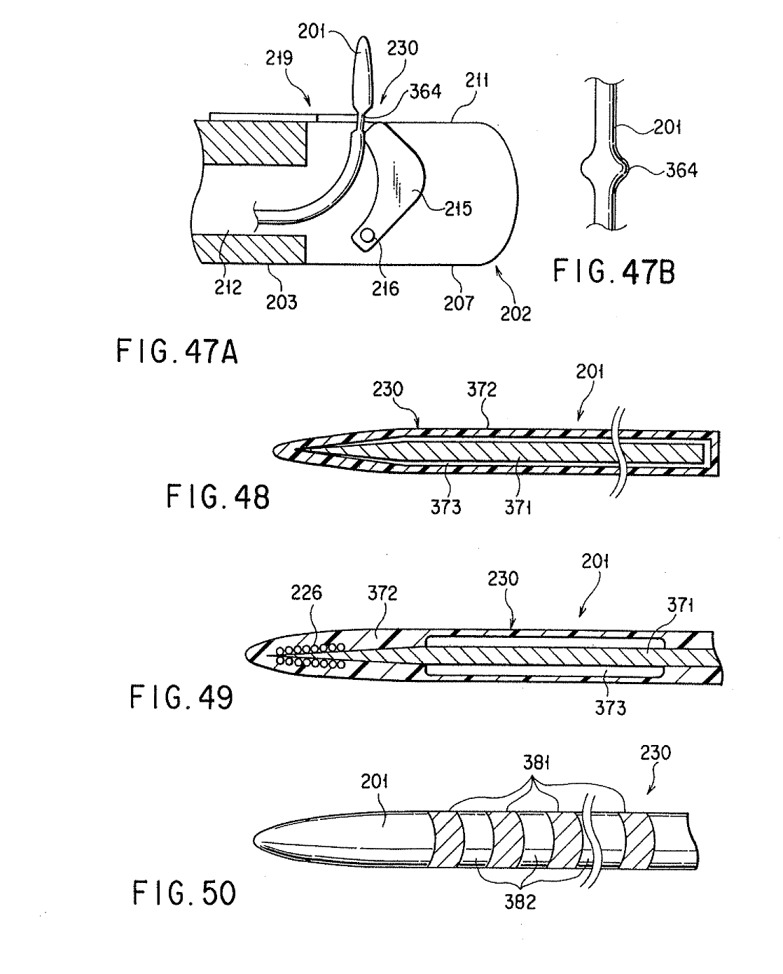

FIGS. 46, 47A and 47B show a twenty-seventh embodiment of the present invention. According to the present embodiment, the configuration of the medical guide wire 201 of the thirteenth embodiment (see FIGS. 22 to 27) is modified in the following manner.

More specifically, according to the present embodiment, the medical guide wire 201 is formed of a wire-shaped core 361 and a coating layer 362 of any of various plastic materials softer than the core 361 that covers the outer surface of the core 361, as shown in FIG. 46.

Further, a small-diameter portion 363 having an outside diameter smaller than that of any other portion is formed on the distal end portion of the core 361. The coating layer 362 is formed so as to be substantially uniform in diameter without excluding the distal end portion of the guide wire 201. Accordingly, a thickened portion 364 of the coating layer 362, having a thickness greater than any other portion, is formed over the small-diameter portion 363 of the core 361, and the thickened portion 364 of the coating layer 362 constitutes the engagement aiding portion 230 of the medical guide wire 201.

In working the medical guide wire 201 of the present embodiment, the guide wire fixing mechanism 223 of the endoscope 202 is caused to engage the thickened portion 364 of the coating layer 362 on the distal end portion of the guide wire 201. When this is done, the forceps raising block 215 on the side of the guide wire fixing mechanism 223 and the contact portion of the guide wire fixture 219 bite into a soft part of the thickened portion 364 of the coating layer 362, as shown in FIG. 47A, thereby ensuring releasable engagement. As this is done, the thickened portion 364 of the coating layer 362 of the guide wire 201 is elastically deformed so as to be squeezed, as shown in FIG. 47B, so that it becomes more catchable, and the area of contact increases. Thus, the guide wire 201 can be prevented from easily moving back and forth, and the engagement between the guide wire 201 and the guide wire fixing mechanism 223 of the endoscope 202 can be aided.

FIG. 48 shows a twenty-eighth embodiment of the present invention. According to the present embodiment, the configuration of the medical guide wire 201 of the thirteenth embodiment (see FIGS. 22 to 27) is modified in the following manner.

More specifically, according to the present embodiment, the medical guide wire 201 is formed of a wire-shaped core 371 and a tubular coating member 372 that surrounds the core 371, as shown in FIG. 48. The coating member 372 is formed of any of various plastic materials that are softer than the core 371. Further, a gap 373 is created between the coating member 372 and the core 371, and the coating member 372 constitutes the engagement aiding portion 230 of the medical guide wire 201. The part of the coating member 372 corresponding to the gap 373 may be hollow or be filled with a filler that is softer than the coating member 372.

In working the medical guide wire 201 of the present embodiment, the guide wire fixing mechanism 223 of the endoscope 202 is caused to engage the distal end portion of the guide wire 201. When this is done, the forceps raising block 215 on the side of the guide wire fixing mechanism 223 and the contact portion of the guide wire fixture 219 bite the coating member 372, thereby ensuring releasable engagement. As this is done, the part of the guide wire 201 corresponding to the coating member 372 is elastically deformed so as to be squeezed, so that it becomes more catchable, and the area of contact increases. Thus, the guide wire 201 can be prevented from easily moving back and forth, and the engagement between the guide wire 201 and the guide wire fixing mechanism 223 of the endoscope 202 can be aided.

FIG. 49 shows a twenty-ninth embodiment of the present invention. According to the present embodiment, the configuration of the medical guide wire 201 of the twenty-eighth embodiment (see FIG. 48) is modified in the following manner.

More specifically, according to the present embodiment, the gap 373 between the coating member 372 and the core 371 of the twenty-eighth embodiment is provided corresponding to the distal end portion of the guide wire 201 alone, and the coating member 372 and the core 371 are intimately in contact with each other in any other portion. That part of the coating member 372 in which the gap 373 is formed constitutes the engagement aiding portion 230 of the medical guide wire 201. The part of the coating member 372 corresponding to the gap 373 may be hollow or be filled with a filler that is softer than the coating member 372.

In working the medical guide wire 201 of the present embodiment, the guide wire fixing mechanism 223 of the endoscope 202 is caused to engage that part of the coating member 372 in which the gap 373 at the distal end of guide wire 201 is formed. When this is done, the forceps raising block 215 on the side of the guide wire fixing mechanism 223 and the contact portion of the guide wire fixture 219 bite the part of the coating member 372 corresponding to the gap 373, thereby ensuring releasable engagement. As this is done, the part of the guide wire 201 corresponding to the coating member 372 is elastically deformed so as to be squeezed, so that it becomes more catchable, and the area of contact increases. Thus, the guide wire 201 can be prevented from easily moving back and forth, and the engagement between the guide wire 201 and the guide wire fixing mechanism 223 of the endoscope 202 can be aided.

FIG. 50 shows a thirtieth embodiment of the present invention. According to the present embodiment, the configuration of the medical guide wire 201 of the thirteenth embodiment (see FIGS. 22 to 27) is modified in the following manner.

More specifically, according to the present embodiment, a plurality of ring-shaped portions 381 of a soft material are juxtaposed substantially at equal spaces, as the engagement aiding portion 230 of the medical guide wire 201, on the distal end portion of the guide wire 201, whereby rigid portions 382 at which the guide wire 201 itself is exposed and the soft ring-shaped portions 381 are arranged alternately in the axial direction, as shown in FIG. 48.

In working the medical guide wire 201 of the present embodiment, the soft ring-shaped portions 381 on the distal end portion of the guide wire 201 can be caused releasably to engage the guide wire fixing mechanism 223 of the endoscope 202 so as to be hooked on it, thereby preventing the guide wire 201 from easily moving back and forth and aiding the engagement between the guide wire 201 and the guide wire fixing mechanism 223 of the endoscope 202.

According to the present embodiment, moreover, the rigid portions 382 at which the guide wire 201 itself is exposed and the soft ring-shaped portions 381 are arranged alternately in the axial direction on the distal end portion of the guide wire 201. In contrast with the case where only a ring-shaped portion 381 is located on the distal end portion of the guide wire 201, therefore, the whole distal end portion of the guide wire 201 can be prevented from becoming easily bendable. Thus, the ease of insertion of the guide wire 201 can be improved.

FIG. 51 shows a thirty-first embodiment of the present invention. According to the present embodiment, the configuration of the medical guide wire 201 of the thirtieth embodiment (see FIG. 50) is modified in the following manner.

More specifically, according to the present embodiment, the medical guide wire 201 is formed of a wire-shaped core 391 and a tubular coating member 392 that surrounds the core 391, as shown in FIG. 51. Further, a plurality of ring-shaped soft portions 393 of a soft material are juxtaposed substantially at equal spaces on the distal end portion of the coating member 392, and a plurality of ring-shaped rigid portions 394 of a rigid material are juxtaposed substantially at equal spaces between the soft portions 393. Thus, the ring-shaped soft portions 393 and the ring-shaped rigid portions 394 are arranged alternately in the axial direction on the distal end portion of the guide wire 201.

In working the medical guide wire 201 of the present embodiment, the ring-shaped soft portions 393 on the distal end portion of the guide wire 201 can be caused releasably to engage the guide wire fixing mechanism 223 of the endoscope 202 so as to be hooked on it, thereby preventing the guide wire 201 from easily moving back and forth and aiding the engagement between the guide wire 201 and the guide wire fixing mechanism 223 of the endoscope 202, as in the case of thirtieth embodiment.

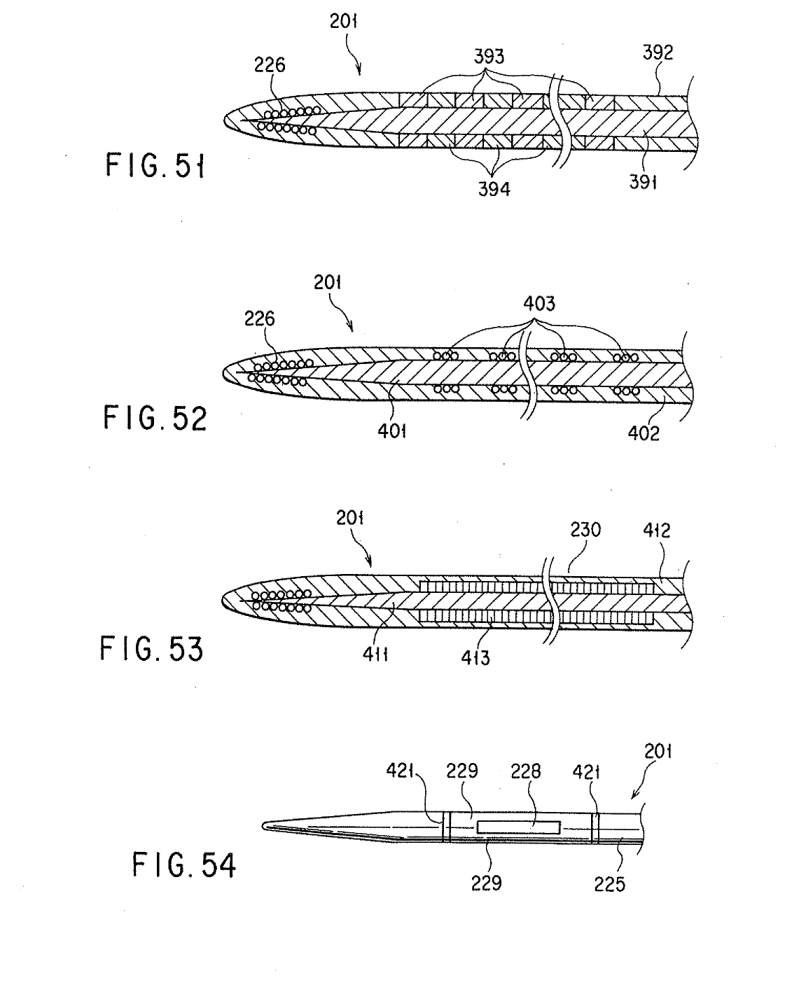

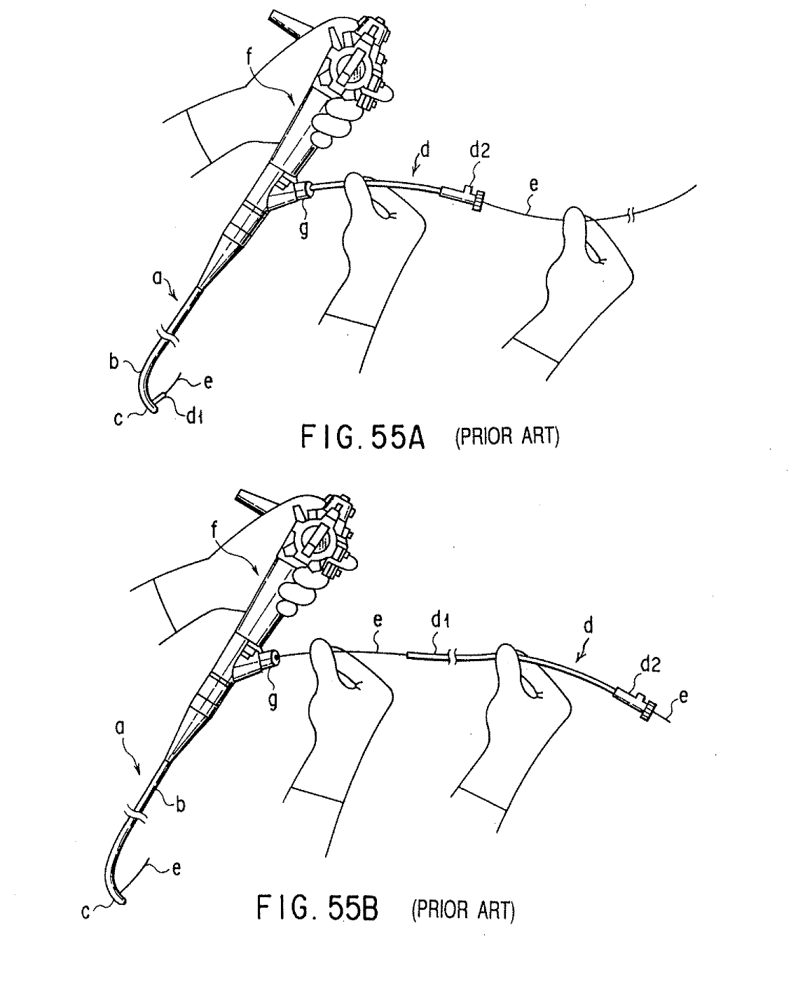

FIG. 52 shows a thirty-second embodiment of the present invention. According to the present embodiment, the configuration of the medical guide wire 201 of the thirtieth embodiment (see FIG. 50) is modified in the following manner.

More specifically, according to the present embodiment, the medical guide wire 201 is formed of a wire-shaped core 401 and a tubular coating member 402 that surrounds the core 401, as shown in FIG. 52. Further, closely-wound coils 403 are wound in a plurality of positions around the distal end portion of the core 401. Thus, those parts of the distal end portion of the guide wire 201 in which the closely-wound coils 403 are wound form a rigid portion, those parts in which the closely-wound coils 403 are not wound form a soft portion, and they are arranged alternately in the axial direction.

In working the medical guide wire 201 of the present embodiment, the coating member 402 in the soft portion of the distal end portion of the guide wire 201 in which the closely-wound coils 403 are not wound can be caused releasably to engage the guide wire fixing mechanism 223 of the endoscope 202 so as to be hooked on it, thereby preventing the guide wire 201 from easily moving back and forth and aiding the engagement between the guide wire 201 and the guide wire fixing mechanism 223 of the endoscope 202, as in the case of thirtieth embodiment.

FIG. 53 shows a thirty-third embodiment of the present invention. According to the present embodiment, the configuration of the medical guide wire 201 of the thirteenth embodiment (see FIGS. 22 to 27) is modified in the following manner.

More specifically, according to the present embodiment, the medical guide wire 201 is formed of a wire-shaped core 411 and a tubular coating member 412 that surrounds the core 411, as shown in FIG. 53. Further, an attraction member 413 that is formed of a magnetic material such as a magnet is attached to the distal end portion of the core 411.

In working the medical guide wire 201 of the present embodiment, the attraction member 413 at the distal end portion of the guide wire 201 can be caused releasably to engage the guide wire fixing mechanism 223 of the endoscope 202 so as to touch it, thereby preventing the guide wire 201 from easily moving back and forth and aiding the engagement between the guide wire 201 and the guide wire fixing mechanism 223 of the endoscope 202.

FIG. 54 shows a thirty-fourth embodiment of the present invention. According to the present embodiment, the configuration of the medical guide wire 201 of the thirteenth embodiment (see FIGS. 22 to 27) is modified in the following manner.

More specifically, according to the present embodiment, marker portions 421, which indicate positions for easy fixation, are provided individually in front and in the rear of that part of the distal end portion of the guide wire sheath 225 of the thirteenth embodiment in which the slits 228 are formed. The marker portions 421 may be visual markers of ink that can be visually recognized in the field of view of the endoscope 202 or X-ray markers that can be recognized by means of X-rays.

In working the medical guide wire 201 of the present embodiment, the positions for easy fixation on the distal end portion of the guide wire 201 can be recognized by checking the marker portions 421 on the distal end portion of the guide wire 201, so that operation for mechanically fixing the guide wire 201 near the distal end portion 207 of the insert section 203 of the endoscope 202 can be carried out securely.

It is to be understood, moreover, that the present invention is not limited to the embodiments described above, and that various modifications may be effected therein without departing from the spirit of the present invention.

### INDUSTRIAL APPLICABILITY

The present invention is effective in the technical field where an endoscope and an appliance such that operation for replacing the appliance is carried out by means of a guide wire are used in combination in endoscopy or endoscopic operations.

1. A guide wire comprising:
a guide wire body to be passed through a channel of an endoscope, the guide wire body having a distal end portion including a distal end and a proximal end portion such that the proximal end can be inserted through a bore which is formed on an appliance and in which the guide wire is to be inserted, to guide the appliance from the proximal end portion to the distal end portion of the guide wire body; and
a retainer extending parallel to the guide wire body along a length between the distal end portion of the guide wire body and the proximal end portion of the guide wire body, the retainer having a distal end portion including a distal end and a proximal end portion, a distal end portion of the retainer being joined at a connection to the distal end portion of the guide wire body to prevent relative movement between the distal end portion of the guide wire body and the distal end portion of the retainer, the proximal end portion of the retainer being retained by an operator, the retainer being arranged outside the bore of the appliance in a state in which the guide wire body is inserted through the bore of the appliance to guide the appliance,
wherein the connection between the guide wire body and retainer is such that the distal ends of each are substantially distal facing.

2. The guide wire according to claim 1, wherein said retainer is a retaining wire.

3. The guide wire according to claim 2, wherein said retaining wire is a resin, a metal or a metal coated with a resin having a stiffness high enough not to be intertwined with said wire.

4. The guide wire according to claim 2, wherein said retaining wire has a separable junction with said guide wire body.

5. The guide wire according to claim 2, wherein said guide wire body is provided with a soft coupling member of an elastic material formed on the distal end portion thereof, the distal end portion of said retaining wire being removably coupled to the coupling member.

6-7. (canceled)

8. The guide wire according to claim 2, wherein said guide wire body and the retaining wire are provided with insulating coating layers, individually.

9. The guide wire according to claim 2, wherein said retaining wire is a ribbon-shaped wire having a substantially flat sectional shape.

10. The guide wire according to claim 2, wherein the proximal end portion of said retaining wire is provided with a retaining portion having a diameter larger than that of any other portion.

11-12. (canceled)

13. The guide wire according to claim 2, wherein the length of said retaining wire is adjusted to 1,000 mm to 2,000 mm.

14. The guide wire according to claim 2, wherein the length of said guide wire body is adjusted to 3,500 mm or less.

15-16. (canceled)
